SEQUENCE LISTING (106 sequences)

SEQ ID NO: 1 - protein, 293 aa, Escherichia coli
MLSPSSVNLGCSWNSLTRNLTSPDSRILSSVRDAAASSDNGAQVKVGNRTYRVVVTDNKFCVTRESHSGCFTNLLHRLGWPKGEISRKIEVMLNSSPVNRAMERGAVHSNRPDLPPVDYAPPELPSVDYNSLPVPGNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 2 - protein, 157 aa, Escherichia coli
NVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 3 - protein, 50 aa, Escherichia coli
NVLYDRTRNEFNPIDISSYNISERSWSENQIMQSYHGGKQDLISVVLSKI

SEQ ID NO: 4 - protein, 303 aa, Escherichia coli
MLSPSSINLGCSWNSLTRNLTSPDNRVLSSVRDAAVHSDSGTQVTVGNRTYRVVVTDNKFCVTRESHSGCFTNLLHRLGWPKGEISRKIEAMLNTSPVSTTIERGSVHSNRPDLPPVDYAQPELPPADYTQSELPRVSNNKSPVPGNVIGKGGNAVVYEDMEDTTKVLKMFTISQSHEEVTSEVRCFNQYYGSGSAEKIYNDNGNVIGIRMNKINGESLLDIPSLPAQAEQAIYDMFDRLEKKGILFVDTTETNVLYDRMRNEFNPIDISSYNVSDISWSEHQVMQSYHGGKLDLISVVLSKI

SEQ ID NO: 5 - protein, 157 aa, Escherichia coli
NVIGKGGNAVVYEDMEDTTKVLKMFTISQSHEEVTSEVRCFNQYYGSGSAEKIYNDNGNVIGIRMNKINGESLLDIPSLPAQAEQAIYDMFDRLEKKGILFVDTTETNVLYDRMRNEFNPIDISSYNVSDISWSEHQVMQSYHGGKLDLISVVLSKI

SEQ ID NO: 6 - protein, 50 aa, Escherichia coli
NVLYDRMRNEFNPIDISSYNVSDISWSEHQVMQSYHGGKLDLISVVLSKI

SEQ ID NO: 7 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 8 - protein, 148 aa, Pseudomonas aeruginosa
MLRKLAAVSLLSLLSAPLLAAECSVDIQGNDQMQFNTNAITVDKSCKQFTVNLSHPGNLPKNVMGHNWVLSTAADMQGVVTDGMASGLDKDYLKPDDSRVIAHTKLIGSGEKDSVTFDVSKLKEGEQYMFFCTFPGHSALMKGTLTLK

SEQ ID NO: 9 - protein, 28 aa, Pseudomonas aeruginosa
LSTAADMQGVVTDGMASGLDKDYLKPDD

SEQ ID NO: 10 - protein, 331 aa, Artificial Sequence
  Description of Artificial Sequence Synthetic polypeptide
MLSTAADMQGVVTDGMASGLDKDYLKPDDPRENFGSEFMLSPSSVNLGCSWNSLTRNLTSPDSRILSSVRDAAASSDNGAQVKVGNRTYRVVVTDNKFCVTRESHSGCFTNLLHRLGWPKGEISRKIEVMLNSSPVNRAMERGAVHSNRPDLPPVDYAPPELPSVDYNSLPVPGNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 11 - protein, 237 aa, Homo sapiens
MNIFDRKINFDALLKFSHITPSTQQHLKKVYASFALCMFVAAAGAYVHMVTHFIQAGLLSALGSLILMIWLMATPHSHETEQKRLGLLAGFAFLTGVGLGPALEFCIAVNPSILPTAFMGTAMIFTCFTLSALYARRRSYLFLGGILMSALSLLLLSSLGNVFFGSIWLFQANLYVGLVVMCGFVLFDTQLIIEKAEHGDQDYIWHCIDLFLDFITVFRKLMMILAMNEKDKKKEKK

SEQ ID NO: 12 - protein, 40 aa, Homo sapiens
MNIFDRKINFDALLKFSHITPSTQQHLKKVYASFALCMFV

SEQ ID NO: 13 - protein, 29 aa, Homo sapiens
MNIFDRKINFDALLKFSHITPSTQQHLKK

SEQ ID NO: 14 - protein, 426 aa, Salmonella bongori
MNISSSGINISTIPTQVKKSVETIRERTKNWFSSEIISVKNTPIFLNEKFKIGKDSPIEFALPQKIKEFFHPKDKNTLNKTLITVKNITDTNNTCKKNISEEVASKMTTAFMRKHIANQSYDYNYRVTSADLLSGGVSISANNRLTVSEGKRDLTSPDANTLSSIQSAVSHSTEGAQVAVGNRTYSVVELNNHFHVSQESGDNCLMNFLYRPGWPKGEVTRKIELVMNTPRLEINPVKNKTILDKTPGQDEMPPIPQVDYNATLHKGEIVGKGGDAIVYADKDDETKVLKMFTIPQLHEEVVHEVECFNTYYGKGSAEIIYSNNDISGIKMTRIQGEPVIYAENLPPHAEQAIYDMFDRLERNNILFVDTTETNVLYDRDTNRFNPIDISSYNLKHTDSKDRQDSIIESYICGKSYLINTVLNKIE

SEQ ID NO: 15 - protein, 196 aa, Shigella flexneri
MKITSTIIQTPFPFENNNSHAGIVTEPILGKLIGQGSTAEIFEDVNDSSALYKKYDLIGNQYNEILEMAWQESELFNAFYGDEASVVIQYGGDVYLRMLRVPGTPLSDIDTADIPDNIESLYLQLICKLNELSIIHYDLNTGNMLYDKESESLFPIDFRNIYAEYYAATKKDKEIIDRRLQMRTNDFYSLLNRKYL

SEQ ID NO: 16 - protein, 320 aa, Artificial Sequence
  Description of Artificial Sequence Synthetic polypeptide
MLSTAADMQGVVTDGMASGLDKDYLKPDDSPSSVNLGCSWNSLTRNLTSPDSRILSSVRDAAASSDNGAQVKVGNRTYRVVVTDNKFCVTRESHSGCFTNLLHRLGWPKGEISRKIEVMLNSSPVNRAMERGAVHSNRPDLPPVDYAPPELPSVDYNSLPVPGNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 17 - protein, 157 aa, Artificial Sequence; identical to SEQ ID NO: 2
  Description of Artificial Sequence Synthetic polypeptide

SEQ ID NO: 18 - protein, 80 aa, Artificial Sequence
  Description of Artificial Sequence Synthetic polypeptide
ASMAQAEHAIYDMFDRLEQKGILFIDTTETNVLYDRTRNEFNPIDISSYNISERSWSENQIMQSYHGGKQDLISVVLSKI

SEQ ID NO: 19 - protein, 30 aa, Artificial Sequence
  Description of Artificial Sequence Synthetic polypeptide
ISERSWSENQIMQSYHGGKQDLISVVLSKI

SEQ ID NO: 20 - protein, 22 aa, Artificial Sequence
  Description of Artificial Sequence Synthetic peptide
NQIMQSYHGGKQDLISVVLSKI

SEQ ID NO: 21 - protein, 15 aa, Artificial Sequence
  Description of Artificial Sequence Synthetic peptide
NQIMQSYHGGKQDLI

SEQ ID NO: 22 - protein, 8 aa, Artificial Sequence
  Description of Artificial Sequence Synthetic peptide
NQIMQSYH

SEQ ID NO: 23 - protein, 9 aa, Artificial Sequence
  Description of Artificial Sequence Synthetic peptide
HGGKQDLIS

SEQ ID NO: 24 - protein, 45 aa, Artificial Sequence
  Description of Artificial Sequence Synthetic polypeptide
RTRNEFNPIDISSYNISERSWSENQIMQSYHGGKQDLISVVLSKI

SEQ ID NO: 25 - protein, 60 aa, Artificial Sequence
  Description of Artificial Sequence Synthetic polypeptide
GILFIDTTETNVLYDRTRNEFNPIDISSYNISERSWSENQIMQSYHGGKQDLISVVLSKI

SEQ ID NO: 26 - protein, 293 aa, Artificial Sequence
  Description of Artificial Sequence Synthetic polypeptide
MLSPSSVNLGCSWNSLTRNLTSPDSRILSSVRDAAASSDNGAQVKVGNRTYRVVVTDNKFCVTRESHSGCFTNLLHRLGWPKGEISRKIEVMLNSSPVNRAMERGAVHSNRPDLPPVDYAPPELPSVDYNSLPVPGNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLAKA

SEQ ID NO: 27 - protein, 303 aa, Artificial Sequence
  Description of Artificial Sequence Synthetic polypeptide
MLSPSSINLGCSWNSLTRNLTSPDNRVLSSVRDAAVHSDSGTQVTVGNRTYRVVVTDNKFCVTRESHSGCFTNLLHRLGWPKGEISRKIEAMLNTSPVSTTIERGSVHSNRPDLPPVDYAQPELPPADYTQSELPRVSNNKSPVPGNVIGKGGNAVVYEDMEDTTKVLKMFTISQSHEEVTSEVRCFNQYYGSGSAEKIYNDNGNVIGIRMNKINGESLLDIPSLPAQAEQAIYDMFDRLEKKGILFVDTTETNVLYDRMRNEFNPIDISSYNVSDISWSEHQVMQSYHGGKLDLISVVLAKA

SEQ ID NO: 28 - protein, 9 aa, Artificial Sequence
  Description of Artificial Sequence Synthetic peptide
VATDGMPAG

SEQ ID NO: 29 - protein, 12 aa, Artificial Sequence
  Description of Artificial Sequence Synthetic peptide
AGADNAFLKAGD

SEQ ID NO: 30 - protein, 18 aa, Artificial Sequence
  Description of Artificial Sequence Synthetic peptide
LTKAADMAGVATDGMPAG

SEQ ID NO: 31 - protein, 28 aa, Artificial Sequence
  Description of Artificial Sequence Synthetic peptide
LTKAADMAGVATDGMPAGADNAFLKAGD

SEQ ID NO: 32 - protein, 166 aa, Artificial Sequence
  Description of Artificial Sequence Synthetic polypeptide
VATDGMPAGNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 33 - protein, 166 aa, Artificial Sequence
  Description of Artificial Sequence Synthetic polypeptide
NVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKIVATDGMPAG

SEQ ID NO: 34 - protein, 169 aa, Artificial Sequence
  Description of Artificial Sequence Synthetic polypeptide
AGADNAFLKAGDNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 35 - protein, 169 aa, Artificial Sequence
  Description of Artificial Sequence Synthetic polypeptide
NVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKIAGADNAFLKAGD

SEQ ID NO: 36 - protein, 175 aa, Artificial Sequence
  Description of Artificial Sequence Synthetic polypeptide
LTKAADMAGVATDGMPAGNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 37 - protein, 175 aa, Artificial Sequence
  Description of Artificial Sequence Synthetic polypeptide
NVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKILTKAADMAGVATDGMPAG

SEQ ID NO: 38 - protein, 185 aa, Artificial Sequence
  Description of Artificial Sequence Synthetic polypeptide
LTKAADMAGVATDGMPAGADNAFLKAGDNVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKI

SEQ ID NO: 39 - protein, 185 aa, Artificial Sequence
  Description of Artificial Sequence Synthetic polypeptide
NVIGKGGNAVVYEDAEDATKVLKMFTTSQSNEEVTNEVRCFNQYYGAGSAEKIYGDNGDIIGIRMDKINGESLLNISSLPAQAEHAIYDMFDRLEQKGILFIDTTETNVLYDRTRNEFNPIDISSYNISERSWSENQIMQSYHGGKQDLISVVLSKILTKAADMAGVATDGMPAGADNAFLKAGD

SEQ ID NO: 40 - protein, 89 aa, Artificial Sequence
  Description of Artificial Sequence Synthetic polypeptide
VATDGMPAGASMAQAEHAIYDMFDRLEQKGILFIDTTETNVLYDRTRNEFNPIDISSYNISERSWSENQIMQSYHGGKQDLISVVLSKI

SEQ ID NO: 41 - protein, 89 aa, Artificial Sequence
  Description of Artificial Sequence Synthetic polypeptide
ASMAQAEHAIYDMFDRLEQKGILFIDTTETNVLYDRTRNEFNPIDISSYNISERSWSENQIMQSYHGGKQDLISVVLSKIVATDGMPAG

SEQ ID NO: 42 - protein, 92 aa, Artificial Sequence
  Description of Artificial Sequence Synthetic polypeptide
AGADNAFLKAGDASMAQAEHAIYDMFDRLEQKGILFIDTTETNVLYDRTRNEFNPIDISSYNISERSWSENQIMQSYHGGKQDLISVVLSKI

SEQ ID NO: 43 - protein, 92 aa, Artificial Sequence
  Description of Artificial Sequence Synthetic polypeptide
ASMAQAEHAIYDMFDRLEQKGILFIDTTETNVLYDRTRNEFNPIDISSYNISERSWSENQIMQSYHGGKQDLISVVLSKIAGADNAFLKAGD

SEQ ID NO: 44 - protein, 98 aa, Artificial Sequence
  Description of Artificial Sequence Synthetic polypeptide
LTKAADMAGVATDGMPAGASMAQAEHAIYDMFDRLEQKGILFIDTTETNVLYDRTRNEFNPIDISSYNISERSWSENQIMQSYHGGKQDLISVVLSKI

SEQ ID NO: 45 - protein, 98 aa, Artificial Sequence
  Description of Artificial Sequence Synthetic polypeptide
ASMAQAEHAIYDMFDRLEQKGILFIDTTETNVLYDRTRNEFNPIDISSYNISERSWSENQIMQSYHGGKQDLISVVLSKILTKAADMAGVATDGMPAG

SEQ ID NO: 46 - protein, 108 aa, Artificial Sequence
  Description of Artificial Sequence Synthetic polypeptide
LTKAADMAGVATDGMPAGADNAFLKAGDASMAQAEHAIYDMFDRLEQKGILFIDTTETNVLYDRTRNEFNPIDISSYNISERSWSENQIMQSYHGGKQDLISVVLSKI

SEQ ID NO: 47 - protein, 108 aa, Artificial Sequence
  Description of Artificial Sequence Synthetic polypeptide
ASMAQAEHAIYDMFDRLEQKGILFIDTTETNVLYDRTRNEFNPIDISSYNISERSWSENQIMQSYHGGKQDLISVVLSKILTKAADMAGVATDGMPAGADNAFLKAGD

SEQ ID NO: 48 - protein, 39 aa, Artificial Sequence
  Description of Artificial Sequence Synthetic polypeptide
VATDGMPAGISERSWSENQIMQSYHGGKQDLISVVLSKI

SEQ ID NO: 49 - protein, 39 aa, Artificial Sequence
  Description of Artificial Sequence Synthetic polypeptide
ISERSWSENQIMQSYHGGKQDLISVVLSKIVATDGMPAG

SEQ ID NO: 50 - protein, 42 aa, Artificial Sequence
  Description of Artificial Sequence Synthetic polypeptide
AGADNAFLKAGDISERSWSENQIMQSYHGGKQDLISVVLSKI

SEQ ID NO: 51 - protein, 42 aa, Artificial Sequence
  Description of Artificial Sequence Synthetic polypeptide
ISERSWSENQIMQSYHGGKQDLISVVLSKIAGADNAFLKAGD

SEQ ID NO: 52 - protein, 48 aa, Artificial Sequence
  Description of Artificial Sequence Synthetic polypeptide
LTKAADMAGVATDGMPAGISERSWSENQIMQSYHGGKQDLISVVLSKI

SEQ ID NO: 53 - protein, 48 aa, Artificial Sequence
  Description of Artificial Sequence Synthetic polypeptide
ISERSWSENQIMQSYHGGKQDLISVVLSKILTKAADMAGVATDGMPAG

SEQ ID NO: 54 - protein, 58 aa, Artificial Sequence
  Description of Artificial Sequence Synthetic polypeptide
LTKAADMAGVATDGMPAGADNAFLKAGDISERSWSENQIMQSYHGGKQDLISVVLSKI

SEQ ID NO: 55 - protein, 58 aa, Artificial Sequence
  Description of Artificial Sequence Synthetic polypeptide
ISERSWSENQIMQSYHGGKQDLISVVLSKILTKAADMAGVATDGMPAGADNAFLKAGD

SEQ ID NO: 56 - protein, 31 aa, Artificial Sequence
  Description of Artificial Sequence Synthetic polypeptide
VATDGMPAGNQIMQSYHGGKQDLISVVLSKI

SEQ ID NO: 57 - protein, 31 aa, Artificial Sequence
  Description of Artificial Sequence Synthetic polypeptide
NQIMQSYHGGKQDLISVVLSKIVATDGMPAG

SEQ ID NO: 58 - protein, 34 aa, Artificial Sequence
  Description of Artificial Sequence Synthetic polypeptide
AGADNAFLKAGDNQIMQSYHGGKQDLISVVLSKI

SEQ ID NO: 59 - protein, 34 aa, Artificial Sequence
  Description of Artificial Sequence Synthetic polypeptide
NQIMQSYHGGKQDLISVVLSKIAGADNAFLKAGD

SEQ ID NO: 60 - protein, 40 aa, Artificial Sequence
  Description of Artificial Sequence Synthetic polypeptide
LTKAADMAGVATDGMPAGNQIMQSYHGGKQDLISVVLSKI

SEQ ID NO: 61 - protein, 40 aa, Artificial Sequence
  Description of Artificial Sequence Synthetic polypeptide
NQIMQSYHGGKQDLISVVLSKILTKAADMAGVATDGMPAG

SEQ ID NO: 62 - protein, 50 aa, Artificial Sequence
  Description of Artificial Sequence Synthetic polypeptide
LTKAADMAGVATDGMPAGADNAFLKAGDNQIMQSYHGGKQDLISVVLSKI

SEQ ID NO: 63 - protein, 50 aa, Artificial Sequence
  Description of Artificial Sequence Synthetic polypeptide
NQIMQSYHGGKQDLISVVLSKILTKAADMAGVATDGMPAGADNAFLKAGD

SEQ ID NO: 64 - protein, 24 aa, Artificial Sequence
  Description of Artificial Sequence Synthetic peptide
VATDGMPAGNQIMQSYHGGKQDLI

SEQ ID NO: 65 - protein, 24 aa, Artificial Sequence
  Description of Artificial Sequence Synthetic peptide
NQIMQSYHGGKQDLIVATDGMPAG

SEQ ID NO: 66 - protein, 27 aa, Artificial Sequence
  Description of Artificial Sequence Synthetic peptide
AGADNAFLKAGDNQIMQSYHGGKQDLI

SEQ ID NO: 67 - protein, 27 aa, Artificial Sequence
  Description of Artificial Sequence Synthetic peptide
NQIMQSYHGGKQDLIAGADNAFLKAGD

SEQ ID NO: 68 - protein, 33 aa, Artificial Sequence
  Description of Artificial Sequence Synthetic polypeptide
LTKAADMAGVATDGMPAGNQIMQSYHGGKQDLI

SEQ ID NO: 69 - protein, 33 aa, Artificial Sequence
  Description of Artificial Sequence Synthetic polypeptide
NQIMQSYHGGKQDLILTKAADMAGVATDGMPAG

SEQ ID NO: 70 - protein, 43 aa, Artificial Sequence
  Description of Artificial Sequence Synthetic polypeptide
LTKAADMAGVATDGMPAGADNAFLKAGDNQIMQSYHGGKQDLI

SEQ ID NO: 71 - protein, 43 aa, Artificial Sequence
  Description of Artificial Sequence Synthetic polypeptide
NQIMQSYHGGKQDLILTKAADMAGVATDGMPAGADNAFLKAGD

SEQ ID NO: 72 - protein, 17 aa, Artificial Sequence
  Description of Artificial Sequence Synthetic peptide
VATDGMPAGNQIMQSYH

SEQ ID NO: 73 - protein, 17 aa, Artificial Sequence
  Description of Artificial Sequence Synthetic peptide
NQIMQSYHVATDGMPAG

SEQ ID NO: 74 - protein, 20 aa, Artificial Sequence
  Description of Artificial Sequence Synthetic peptide
AGADNAFLKAGDNQIMQSYH

SEQ ID NO: 75 - protein, 20 aa, Artificial Sequence
  Description of Artificial Sequence Synthetic peptide
NQIMQSYHAGADNAFLKAGD

SEQ ID NO: 76 - protein, 26 aa, Artificial Sequence
  Description of Artificial Sequence Synthetic peptide
LTKAADMAGVATDGMPAGNQIMQSYH

SEQ ID NO: 77 - protein, 26 aa, Artificial Sequence
  Description of Artificial Sequence Synthetic peptide
NQIMQSYHLTKAADMAGVATDGMPAG

SEQ ID NO: 78 - protein, 36 aa, Artificial Sequence
  Description of Artificial Sequence Synthetic polypeptide
LTKAADMAGVATDGMPAGADNAFLKAGDNQIMQSYH

SEQ ID NO: 79 - protein, 36 aa, Artificial Sequence
  Description of Artificial Sequence Synthetic polypeptide
NQIMQSYHLTKAADMAGVATDGMPAGADNAFLKAGD

SEQ ID NO: 80 - protein, 18 aa, Artificial Sequence
  Description of Artificial Sequence Synthetic peptide
VATDGMPAGHGGKQDLIS

SEQ ID NO: 81 - protein, 18 aa, Artificial Sequence
  Description of Artificial Sequence Synthetic peptide
HGGKQDLISVATDGMPAG

SEQ ID NO: 82 - protein, 21 aa, Artificial Sequence
  Description of Artificial Sequence Synthetic peptide
AGADNAFLKAGDHGGKQDLIS

SEQ ID NO: 83 - protein, 21 aa, Artificial Sequence
  Description of Artificial Sequence Synthetic peptide
HGGKQDLISAGADNAFLKAGD

SEQ ID NO: 84 - protein, 27 aa, Artificial Sequence
  Description of Artificial Sequence Synthetic peptide
LTKAADMAGVATDGMPAGHGGKQDLIS

SEQ ID NO: 85 - protein, 27 aa, Artificial Sequence
  Description of Artificial Sequence Synthetic peptide
HGGKQDLISLTKAADMAGVATDGMPAG

SEQ ID NO: 86 - protein, 37 aa, Artificial Sequence
  Description of Artificial Sequence Synthetic polypeptide
LTKAADMAGVATDGMPAGADNAFLKAGDHGGKQDLIS

SEQ ID NO: 87 - protein, 37 aa, Artificial Sequence
  Description of Artificial Sequence Synthetic polypeptide
HGGKQDLISLTKAADMAGVATDGMPAGADNAFLKAGD

SEQ ID NO: 88 - protein, 54 aa, Artificial Sequence
  Description of Artificial Sequence Synthetic polypeptide
VATDGMPAGRTRNEFNPIDISSYNISERSWSENQIMQSYHGGKQDLISVVLSKI

SEQ ID NO: 89 - protein, 54 aa, Artificial Sequence
  Description of Artificial Sequence Synthetic polypeptide
RTRNEFNPIDISSYNISERSWSENQIMQSYHGGKQDLISVVLSKIVATDGMPAG

SEQ ID NO: 90 - protein, 57 aa, Artificial Sequence
  Description of Artificial Sequence Synthetic polypeptide
AGADNAFLKAGDRTRNEFNPIDISSYNISERSWSENQIMQSYHGGKQDLISVVLSKI

SEQ ID NO: 91 - protein, 57 aa, Artificial Sequence
  Description of Artificial Sequence Synthetic polypeptide
RTRNEFNPIDISSYNISERSWSENQIMQSYHGGKQDLISVVLSKIAGADNAFLKAGD

SEQ ID NO: 92 - protein, 63 aa, Artificial Sequence
  Description of Artificial Sequence Synthetic polypeptide
LTKAADMAGVATDGMPAGRTRNEFNPIDISSYNISERSWSENQIMQSYHGGKQDLISVVLSKI

SEQ ID NO: 93 - protein, 63 aa, Artificial Sequence
  Description of Artificial Sequence Synthetic polypeptide
RTRNEFNPIDISSYNISERSWSENQIMQSYHGGKQDLISVVLSKILTKAADMAGVATDGMPAG

SEQ ID NO: 94 - protein, 73 aa, Artificial Sequence
  Description of Artificial Sequence Synthetic polypeptide
LTKAADMAGVATDGMPAGADNAFLKAGDRTRNEFNPIDISSYNISERSWSENQIMQSYHGGKQDLISVVLSKI

SEQ ID NO: 95 - protein, 73 aa, Artificial Sequence
  Description of Artificial Sequence Synthetic polypeptide
RTRNEFNPIDISSYNISERSWSENQIMQSYHGGKQDLISVVLSKILTKAADMAGVATDGMPAGADNAFLKAGD

SEQ ID NO: 96 - protein, 69 aa, Artificial Sequence
  Description of Artificial Sequence Synthetic polypeptide
VATDGMPAGGILFIDTTETNVLYDRTRNEFNPIDISSYNISERSWSENQIMQSYHGGKQDLISVVLSKI

SEQ ID NO: 97 - protein, 69 aa, Artificial Sequence
  Description of Artificial Sequence Synthetic polypeptide
GILFIDTTETNVLYDRTRNEFNPIDISSYNISERSWSENQIMQSYHGGKQDLISVVLSKIVATDGMPAG

SEQ ID NO: 98 - protein, 72 aa, Artificial Sequence
  Description of Artificial Sequence Synthetic polypeptide
AGADNAFLKAGDGILFIDTTETNVLYDRTRNEFNPIDISSYNISERSWSENQIMQSYHGGKQDLISVVLSKI

SEQ ID NO: 99 - protein, 72 aa, Artificial Sequence
  Description of Artificial Sequence Synthetic polypeptide
GILFIDTTETNVLYDRTRNEFNPIDISSYNISERSWSENQIMQSYHGGKQDLISVVLSKIAGADNAFLKAGD

SEQ ID NO: 100 - protein, 78 aa, Artificial Sequence
  Description of Artificial Sequence Synthetic polypeptide
LTKAADMAGVATDGMPAGGILFIDTTETNVLYDRTRNEFNPIDISSYNISERSWSENQIMQSYHGGKQDLISVVLSKI

SEQ ID NO: 101 - protein, 78 aa, Artificial Sequence
  Description of Artificial Sequence Synthetic polypeptide
GILFIDTTETNVLYDRTRNEFNPIDISSYNISERSWSENQIMQSYHGGKQDLISVVLSKILTKAADMAGVATDGMPAG

SEQ ID NO: 102 - protein, 88 aa, Artificial Sequence
  Description of Artificial Sequence Synthetic polypeptide
LTKAADMAGVATDGMPAGADNAFLKAGDGILFIDTTETNVLYDRTRNEFNPIDISSYNISERSWSENQIMQSYHGGKQDLISVVLSKI

SEQ ID NO: 103 - protein, 88 aa, Artificial Sequence
  Description of Artificial Sequence Synthetic polypeptide
GILFIDTTETNVLYDRTRNEFNPIDISSYNISERSWSENQIMQSYHGGKQDLISVVLSKILTKAADMAGVATDGMPAGADNAFLKAGD

SEQ ID NO: 104 - protein, 13 aa, Human immunodeficiency virus 1
GRKKRRQRRRPPQ

SEQ ID NO: 105 - protein, 43 aa, Artificial Sequence
  Description of Artificial Sequence Synthetic polypeptide
ISERSWSENQIMQSYHGGKQDLISVVLSKIGRKKRRQRRRPPQ

SEQ ID NO: 106 - protein, 93 aa, Artificial Sequence
  Description of Artificial Sequence Synthetic polypeptide
ASMAQAEHAIYDMFDRLEQKGILFIDTTETNVLYDRTRNEFNPIDISSYNISERSWSENQIMQSYHGGKQDLISVVLSKIGRKKRRQRRRPPQ

(19) United States
(12) Patent Application Publication
(10) Pub. No.: US 2021/0361742 A1
(43) Pub. Date: Nov. 25, 2021

(54) PEPTIDE THERAPEUTICS FOR THE TREATMENT OF CANCER AND USES THEREOF

(71) Applicant: MicroQuin Ltd., Cambridge, MA (US)

(72) Inventors: Keith Scott ROBINSON, London (GB); Wei LUO, Singapore (SG)

(21) Appl. No.: 17/272,160

(22) PCT Filed: Aug. 27, 2019
(86) PCT No.: PCT/US19/48414

§ 371 Date: Feb. 26, 2021

Related U.S. Application Data
(60) Provisional application No. 62/723,428, filed on Aug. 27, 2018.

(51) Int. Cl. A61K 38/17; C07K 14/47; A61K 45/06; A61P 35/00

(52) U.S. Cl. CPC A61K 38/1761; C07K 14/4747; A61K 38/00; A61P 35/00; C07K 2319/30; A61K 45/06

(57) ABSTRACT

The disclosure provides BI-1 modulating peptides and methods for treating cancer in a subject by administering an effective amount of a BI-1 modulating peptide.

### 1. CROSS-REFERENCE TO RELATED APPLICATIONS

This application claims priority to U.S. Provisional Application No. 62/723,428, filed Aug. 27, 2018, which is hereby incorporated by reference in its entirety.

### 2. SEQUENCE LISTING

The instant application contains a Sequence Listing which has been submitted via EFS-Web and is hereby incorporated by reference in its entirety. Said ASCII copy, created on Month XX, 20XX, is named XXXXXUS_sequencelisting.txt, and is X,XXX,XXX bytes in size.

### 3. BACKGROUND

Bax Inhibitor-1 (Bax-1) has been shown to have diverse roles inside cells regulating apoptosis, ER stress, production of reactive oxygen species (ROS), actin cytoskeletal dynamics, and cytosolic calcium levels (Robinson et al., Oncogene 30: 2391-2400, 2011). Expression of BI-1 differs significantly across human cancer types, with the protein being highly expressed in breast, glioma, prostate, uterine and ovarian cancers but downregulated in stomach, colon, kidney, lung, and rectal cancers (Grzmil et al., J Pathol 208: 340-349, 2006; Schmits et al., Int J Cancer 98: 73-77, 2002; and del Carmen Garcia Molina Wolgien et al, Eur J Gynaecol Oncol 26: 501-504, 2005).

Prior studies using RNA interference (RNAi) to knock down BI-1 expression in breast and prostate cancer cells resulted in spontaneous apoptosis in some but not all cell lines, indicating that BI-1 is essential for cancer survival in some cancer subtypes (Grzmil et al., J Pathol 208: 340-349, 2006; Grzmil et al., Am J Pathol 163: 543-552, 2003; Lima et al., Cancer Gene Ther 11: 309-316, 2004). Cells that did not undergo spontaneous apoptosis following BI-1 knock down by RNAi showed signs of cellular stress and were highly sensitized to apoptotic induction. BI-1 thus presents as a unique but as-yet untested target candidate for a cancer therapeutic.

### 4. SUMMARY

Disclosed herein are Bax Inhibitor-1 (BI-1) modulating peptides comprising a BI-1 modulating domain. In some embodiments the BI-1 modulating peptide comprises a targeting domain capable of conferring on the BI-1 modulating peptide the ability to cross a mammalian cell plasma membrane. These BI-1 modulating peptides can be used for treating cancer.

In some embodiments, the BI-1 modulating domain comprises a peptide segment having the sequence of SEQ ID NO: 22 or a sequence that differs by no more than one amino acid residue from the sequence of SEQ ID NO: 22; and/or a peptide segment having the sequence of SEQ ID NO: 23 or a sequence that differs by no more than one amino acid residue from the sequence of SEQ ID NO: 23. In some embodiments, the BI-1 modulating domain comprises a peptide segment having the amino acid of SEQ ID NO: 22 and/or SEQ ID NO: 23. In specific embodiments, the BI-1 modulating domain comprises a peptide segment having the sequence of SEQ ID NO: 22 and a peptide segment having the sequence of SEQ ID NO: 23. In specific embodiments, the peptide segment having the sequence of SEQ ID NO: 22 is amino terminal to the segment having the sequence of SEQ ID NO: 23. In particular embodiments, the sequence of SEQ ID NO:22 and SEQ ID NO:23 overlap within the segment.

In some embodiments, the BI-1 modulating domain has the sequence of SEQ ID NO: 16. In some embodiments, the BI-1 modulating domain has the sequence of SEQ ID NO: 17. In some embodiments, the BI-1 modulating domain has the sequence of SEQ ID NO: 18. In some embodiments, the BI-1 modulating domain has the sequence of SEQ ID NO: 19. In some embodiments, the BI-1 modulating domain has the sequence of SEQ ID NO: 20. In some embodiments, the BI-1 modulating domain has the sequence of SEQ ID NO: 21. In some embodiments, the BI-1 modulating domain has the sequence of SEQ ID NO: 24. In some embodiments, the BI-1 modulating domain has the sequence of SEQ ID NO: 25. In some embodiments, the BI-1 modulating domain has the sequence of SEQ ID NO: 26. In some embodiments, the BI-1 modulating domain has the sequence of SEQ ID NO: 27.

In some embodiments, the BI-1 modulating domain is capable of binding to a BI-1 protein. In some embodiments, the BI-1 modulating domain is capable of binding to a site within a BI-1 protein within the amino acid sequence of SEQ ID NO: 13.

In some embodiments, the BI-1 modulating peptide is capable of being coupled to a liposome. In some embodiments, the peptide is capable of being conjugated to a nanoparticle.

In some embodiments, the targeting domain is a cell penetrating peptide (CPP). In some embodiments, the targeting domain is an antibody or a fragment of an antibody. In some embodiments, the targeting domain is capable of binding a tumor-associated antigen. In particular embodiments, the targeting domain is at the amino terminus of the BI-1 modulating peptide. In particular embodiments, the targeting domain is at the carboxy terminus of the peptide.

In some embodiments, the BI-1 modulating peptide is between 5 and 400 amino acids in length. In some embodiments, the BI-1 modulating peptide is between 8 and 40 amino acids in length. In some embodiments, the BI-1 modulating peptide is between 15 and 45 amino acids in length. In some embodiments, the BI-1 modulating peptide is between 22 and 50 amino acids in length. In some embodiments, the BI-1 modulating peptide is between 30 and 60 amino acids in length. In some embodiments, the BI-1 modulating peptide is between 45 and 75 amino acids in length. In some embodiments, the BI-1 modulating peptide is between 6 and 100 amino acids in length. In some embodiments, the BI-1 modulating peptide is between 80 and 110 amino acids in length. In some embodiments, the BI-1 modulating peptide is between 280 and 320 amino acids in length.

In some embodiments, the BI-1 modulating peptide has an amino acid sequence with at least 85% sequence identity to the sequence of any one of SEQ ID NOs: 19-23 and 48-87. In some embodiments, the BI-1 modulating peptide has an amino acid sequence with at least 85% sequence identity to the sequence of SEQ ID NO: 19. In some embodiments, the peptide has an amino acid sequence with at least 85% sequence identity to the sequence of SEQ ID NO: 20. In some embodiments, the peptide has an amino acid sequence with at least 85% sequence identity to the sequence of SEQ ID NO: 21. In some embodiments, the peptide has an amino acid sequence with at least 85% sequence identity to the sequence of SEQ ID NO: 22. In some embodiments, the peptide has an amino acid sequence with at least 85% sequence identity to the sequence of SEQ ID NO: 23.

In some embodiments, the BI-1 modulating peptide comprises a chemical modification. In some embodiments, the chemical modification is phosphorylation, glycosylation, and/or lipidation. In some embodiments, the chemical modification is a covalent linkage of a fatty acid. In some embodiments, the chemical modification is a chemical blocking of the terminal amine group of the peptide. In some embodiments, the chemical modification is a chemical blocking of the terminal carboxy group of the peptide.

In some embodiments, the BI-1 modulating peptide further comprises an Fc polypeptide or domain. In some embodiments, the peptide further comprises a non-peptide linker. In some embodiments, the peptide is conjugated to one or more PEG molecules.

In certain embodiments, the BI-1 modulating peptide is capable of passing through a plasma membrane of a mammalian cell. In some embodiments the mammalian cell is a human cell.

In another aspect, provided herein is a pharmaceutical composition comprising the BI-1 modulating peptide and a pharmaceutically acceptable carrier. In some embodiments, the pharmaceutical composition is suitable for parenteral administration. In some embodiments, the pharmaceutical composition is suitable for intravenous administration. In some embodiments, the pharmaceutical composition is suitable for subcutaneous administration.

In some embodiments, the concentration of active ingredient in the pharmaceutical composition is 100 nM or greater.

In some embodiments, the pharmaceutical composition is in a single-dose prefilled syringe.

In some embodiments, the pharmaceutical composition comprises a pharmaceutically acceptable carrier suitable for enhancing solubility of the BI-1 modulating peptide.

In another aspect, provided herein is a method of treating a proliferative disease in a patient, comprising administering to the subject an effective amount of the BI-1 modulating peptide or the pharmaceutical composition comprising the BI-1 modulating peptide. In some embodiments, the proliferative disease is cancer. In some embodiments, the cancer is at least one of breast, ovarian, lung, uterine, and colon cancer. In some embodiments, the cancer is breast cancer.

In some embodiments, administering the BI-1 modulating peptide or the pharmaceutical composition comprising the BI-1 modulating peptide results in cytosolic calcium levels in cells of the subject. In some embodiments, administering the peptide or pharmaceutical composition comprising the peptide results in an increase in cytosolic concentration of ions in cells of the subject. In some embodiments, the administering results in an increase in permeabilization of mitochondrial membranes in neoplastic cells in the subject.

In some embodiments, administering the BI-1 modulating peptide or the pharmaceutical composition comprising the BI-1 modulating peptide induces death of neoplastic cells in the subject. In some embodiments, the administering induces apoptosis and/or paraptosis of neoplastic cells in the subject.

In some embodiments, the BI-1 modulating peptide or the pharmaceutical composition comprising the BI-1 modulating peptide is administered to the subject by intravenous administration. In some embodiments, the peptide or pharmaceutical composition is administered by subcutaneous administration. In some embodiments, the peptide or pharmaceutical composition is administered by intrathecal or intra-cisterna magna administration.

In some embodiments, the method further comprises administering a second effective amount of a further treatment. In specific embodiments, the further treatment comprises a chemotherapeutic agent, a radiation treatment, or an antibody or antibody fragment.

In some embodiments, the subject that is administered the BI-1 modulating peptide or the pharmaceutical composition comprising the BI-1 modulating is a mammal. In specific embodiments, the subject is a human.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A, FIG. 1B, FIG. 1C, FIG. 1D, and FIG. 1E show that MQ001 and MQ002 interact with BI-1. FIG. 1A shows immunoblot results following co-immunoprecipitation of HA-tagged MQ001 and MQ002 with BI-1; FIG. 1B shows results of a yeast two-hybrid analysis confirming interaction between BI-1 and MQ001 and BI-1 and MQ002; FIG. 1C shows results of a yeast two-hybrid analysis confirming interaction between the N-terminus of BI-1 and MQ001 and the N-terminus of BI-1 and MQ002; FIG. 1D presents results of a β-galactosidase assay showing interaction of MQ001 and BI-1, and MQ001 and the N-terminal 40 amino acids of BI-1; FIG. 1E presents immunofluorescence images of HeLa cells co-transfected with either HA-tagged MQ001 or HA-tagged MQ002 and Myc-tagged BI-1, showing co-localization of MQ001 and BI-1 and co-localization of MQ002 and BI-1.

FIG. 2A, FIG. 2B, and FIG. 2C show that MQ001 and MQ002 selectively induce cell death in human breast cancer cells. Graphs in FIG. 2A show the percentage of cells with condensed nuclei and external annexin-v in MCF-7 breast cancer cells treated with MQ001 and MQ002 compared with MCF-10F cells derived from normal breast tissue; FIG. 2B shows flow cytometry analysis of MCF-7 cells treated with MQ001 and GFP-CPP (control); FIG. 2C presents results of a MTT cell viability assay on MCF-7 and MCF-10F cells treated with MQ001 and MQ002.

FIG. 3A, FIG. 3B, and FIG. 3C present data showing that MQ001 and MQ002 induce cell death in several breast cancer subtypes and that BI-1 is important for the therapeutic effects of the peptides on breast cancer cells. FIG. 3A presents graphs showing that an increase in condensed nuclei was seen in all breast cancer cell lines tested in response to treatment with MQ001 and MQ002; FIG. 3B shows that the seven breast cancer cell lines tested represent three different breast cancer subtypes; FIG. 3C shows results of siRNA knockdown of BI-1, demonstrating that MQ001 and MQ002 do not induce cell death in breast cancer cells in the absence of BI-1.

FIG. 4 presents graphs showing the percentage of cells with externalized annexin-v in seven breast cancer cell lines in response to treatment with MQ001 and MQ002.

FIG. 5 shows quantified cell death data correlated with phase-contrast microscopy images of MCF-7 cells treated with MQ70C, showing that MQ70C induces cell death in a dose-dependent manner.

FIG. 6A and FIG. 6B present phase-contrast microscopy images of MCF-7 cells following treatment with various BI-1 modulating peptides, showing that all of the peptides tested induce cell death in MCF-7 cells.

FIG. 7A, FIG. 7B, FIG. 7C, FIG. 7D, FIG. 7E, FIG. 7F, FIG. 7G, FIG. 7H, and FIG. 7I present results demonstrating that the BI-1 modulating peptides MQ001 and MQ002 induce cell death in cancer cells other than breast cancer. FIG. 7A shows the percentage of lung cancer and breast cancer cells with condensed nuclei following treatment with MQ001 and MQ002. FIG. 7B and FIG. 7C show phase-contrast microscopy images of lung cancer and colon cancer cells treated with the BI-1 modulating peptide MQ30C over time. FIG. 7D shows immunofluorescence images stained to assess lysosomes and mitochondrial membranes in ovarian cancer cells following treatment with MQ16. FIG. 7E shows dose response curves of MQ16 treatment in each of four ovarian cancer cell lines. FIG. 7F shows calcium efflux in ovarian cancer cells following treatment with various BI-1 modulating peptides. FIG. 7G presents immunofluorescence images showing formation of lysosomes and permeabilization of mitochondrial membranes in uterine cancer cells following treatment with MQ16. FIG. 7H shows dose response curves of MQ16 treatment in each of five uterine cancer cell lines. FIG. 7I shows calcium efflux in uterine cancer cells in response to treatment with various BI-1 modulating peptides.

FIG. 8A and FIG. 8B show the percentage of MCF-7 cells with cleaved caspase 3 following treatment with MQ001, MQ002 and intrinsic and extrinsic inducers of apotosis. Results shown in FIG. 8A demonstrate that MQ001 and MQ002 have an anti-apoptotic effect on MCF-7 cells subjected to intrinsic inducers of apoptosis but do not have the same protective effect on cells exposed to an extrinsic inducer of apoptosis. Results shown in FIG. 8B further demonstrate that BI-1 expression is necessary for the anti-apoptotic effects of MQ001 and MQ002.

FIG. 9A and FIG. 9B show the percentage of non-cancerous cells with condensed nuclei and external annexin-v following treatment with MQ001 and MQ002.

FIG. 10A, FIG. 10B, and FIG. 10C show the relative change in cytoplasmic calcium concentration in cells following treatment with MQ001 or MQ002 (FIG. 10A), in cells overexpressing BI-1 (FIG. 10B), and following treatment with various BI-1 modulating peptides (FIG. 10C).

FIG. 11A and FIG. 11B show changes in cytosolic ROS levels following treatment with MQ001 and MQ002 (FIG. 11A) and changes in cell morphology and staining following treatment with MQ16 (FIG. 11B).

FIG. 12 presents immunofluorescence images of MCF-10F and MCF-7 cells treated with HA-tagged MQ001 or HA-tagged MQ002 and stained for viable mitochondria.

FIG. 13A, FIG. 13B, and FIG. 13C present immunofluorescence and phase-state microscopy images of cells treated with MQ001 and MQ002, showing changes in ER morphology following treatment (FIG. 13A) and disruption of actin localization following treatment (FIG. 13B and FIG. 13C).

FIG. 14A, FIG. 14B, and FIG. 14C present immunofluorescence microscopy images of cells stained with markers for ER (FIG. 14A), autophagy proteins (FIG. 14B), and lysosomes (FIG. 14C).

FIG. 15A, FIG. 15B, FIG. 15C, and FIG. 15D show immunoblots evaluating phospho-JNK and phospho-ERK expression in MCF-10F and MCF-7 cells following treatment with MQ001 and MQ002 (FIG. 15A), gel electrophoresis evaluating RT-PCR results of BCL-2 family members and the UPR induced transcription factor CHOP (FIG. 15B), immunoblots evaluating phospho-Bcl-2 expression in MCF-10F and MCF-7 cells following treatment with MQ001 and MQ002 (FIG. 15C), and quantification of ER disruption (white bars) overlaid onto counts of cells with nuclear condensation (black bars) following treatment with MQ001 or MQ002, compared to control (FIG. 15D).

FIG. 16A and FIG. 16B present graphs showing changes in tumor volume (FIG. 16A) and body weight (FIG. 16B) in mouse models of human breast cancer following treatment with MQ001.

FIG. 17 shows results of H & E staining evaluating toxicity of MQ001 in a mouse model of human breast cancer.

FIG. 18A and FIG. 18B show results of stability assessments of MQ001 in plasma (FIG. 18A) and microsomes (FIG. 18B).

### 6. DETAILED DESCRIPTION OF THE INVENTION

### Definitions

Unless defined otherwise, all technical and scientific terms used herein have the meaning commonly understood by one of ordinary skill in the art to which the invention pertains.

The term “amino acid” refers to natural amino acids, unnatural amino acids, and amino acid analogs. Unless otherwise indicated, the term “amino acid” includes both D and L stereoisomers if the respective structure allows such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids, or non-natural amino acid include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), Nalkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “mammal” as used herein includes both humans and non-humans and includes but is not limited to humans, non-human primates, canines, felines, murines, bovines, equines, and porcines.

As used herein, the term “peptide” refers to a polymer of amino acids linked together by peptide bonds. A peptide can comprise natural amino acids, non-natural amino acids, amino acid analogs, and/or modified amino acids. A peptide can be a portion or fragment of naturally occurring protein or a non-natural (synthetic) protein or polypeptide.

As used herein, the term “mutant peptide” refers to a variant of a naturally occurring peptide having a distinct amino acid sequence from the most common variant occurring in nature, referred to as the “wild-type” sequence. A mutant peptide can comprise one or more amino acid substitution, deletion, or insertion as compared to the wild-type peptide.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues can be divided into classes based on common side group properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid having a common side group property.

In some embodiments, unless otherwise specified, a conservative or semi-conservative amino acid substitution can also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein include natural amino acids, non-natural amino acids, and amino acid analogs. For example, nor-leucine can be used to substitute methionine.

Non-conservative substitutions can involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

As used herein, the term “subject” broadly refers to any animal, including but not limited to, human and non-human animals (e.g., dogs, cats, cows, horses, sheep, pigs, poultry, fish, crustaceans, etc.). As used herein, the term “patient” refers to a human subject.

Unless otherwise specified, “BI-1 modulating peptide” refers to a peptide that interacts with Bax Inhibitor-1 protein (BI-1). A BI-1 modulating peptide may inhibit or stimulate BI-1 activity. A given BI-1 modulating peptide may inhibit BI-1 under particular conditions in some cells and may stimulate BI-1 in other cells. A BI-1 modulating peptide may directly bind to BI-1 via one or more amino acid residues. A BI-1 modulating peptide may interact with and modulate BI-1 indirectly, including via one or more signaling molecules.

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

The term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient (e.g., isolated BI-1 modulating peptide) with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21th Ed., Mack Publ. Co., Easton, Pa. (2005), incorporated herein by reference in its entirety.

It must be noted that, as used in the specification and the appended claims, the singular forms “a,” “an” and “the” include plural referents unless the context clearly dictates otherwise.

6.1. BI-1 Modulating Peptides

In a first aspect, disclosed herein is an isolated BI-1 modulating peptide.

In various embodiments, the isolated BI-1 modulating peptide is no more than 320 amino acids in length. In certain embodiments, the isolated BI-1 modulating peptide is no more than 340 amino acids in length. In certain embodiments, the isolated BI-1 modulating peptide is no more than 320 amino acids in length. In certain embodiments, the isolated BI-1 modulating peptide is no more than 310 amino acids in length. In certain embodiments, the isolated BI-1 modulating peptide is no more than 300 amino acids in length. In certain embodiments, the isolated BI-1 modulating peptide is no more than 250 amino acids in length. In certain embodiments, the isolated BI-1 modulating peptide is no more than 200 amino acids in length. In certain embodiments, the isolated BI-1 modulating peptide is no more than 175 amino acids in length. In certain embodiments, the isolated BI-1 modulating peptide is no more than 150 amino acids in length. In certain embodiments, the isolated BI-1 modulating peptide is no more than 125 amino acids in length. In certain embodiments, the isolated BI-1 modulating peptide is no more than 100 amino acids in length. In certain embodiments, the isolated BI-1 modulating peptide is no more than 80 amino acids in length. In certain embodiments, the isolated BI-1 modulating peptide is no more than 70 amino acids in length. In certain embodiments, the isolated BI-1 modulating peptide is no more than 60 amino acids in length. In certain embodiments, the isolated BI-1 modulating peptide is no more than 50 amino acids in length. In certain embodiments, the isolated BI-1 modulating peptide is no more than 40 amino acids in length. In certain embodiments, the isolated BI-1 modulating peptide is no more than 30 amino acids in length. In certain embodiments, the isolated BI-1 modulating peptide is no more than 25 amino acids in length. In certain embodiments, the isolated BI-1 modulating peptide is no more than 20 amino acids in length. In certain embodiments, the isolated BI-1 modulating peptide is no more than 15 amino acids in length. In certain embodiments, the isolated BI-1 modulating peptide is no more than 10 amino acids in length.

The isolated BI-1 modulating peptide comprises a BI-1 modulating domain.

In certain embodiments, the isolated BI-1 modulating peptide further comprises a targeting domain. In some embodiments, the BI-1 modulating peptide further comprises an Fc polypeptide or domain.

6.1.1. BI-1 Modulating Domain

In certain embodiments, the BI-1 modulating domain of the BI-1 modulating peptide comprises one or more binding sites that bind to BI-1. Each of the one or more binding sites comprises one or more amino acid residues. In some embodiments, at least one of the one or more binding sites comprises two or more amino acid residues at adjacent positions. In some embodiments at least one of the one or more binding sites comprises two or more amino acid residues at non-adjacent positions.

In some embodiments the BI-1 modulating domain comprises two or more BI-1 binding sites with different binding affinities.

In some embodiments the BI-1 modulating domain comprises an amino acid sequence having at least 75%, 80%, 85%, 90%, 95%, 96%, 97%, 98%, or 99% sequence identity to SEQ ID NO: 18. In some embodiments the BI-1 modulating domain comprises an amino acid sequence having at least 75%, 80%, 85%, 90%, 95%, 96%, 97%, 98%, or 99% sequence identity to SEQ ID NO: 19. In some embodiments the BI-1 modulating domain comprises an amino acid sequence having at least 75%, 80%, 85%, 90%, 95%, 96%, 97%, 98%, or 99% sequence identity to SEQ ID NO: 20. In some embodiments the BI-1 modulating domain comprises an amino acid sequence having at least 75%, 80%, 85%, 90%, 95%, 96%, 97%, 98%, or 99% sequence identity to SEQ ID NO: 21. In some embodiments the BI-1 modulating domain comprises an amino acid sequence having at least 75%, 80%, 85%, 90%, 95%, 96%, 97%, 98%, or 99% sequence identity to SEQ ID NO: 22. In some embodiments the BI-1 modulating domain comprises an amino acid sequence having at least 75%, 80%, 85%, 90%, 95%, 96%, 97%, 98%, or 99% sequence identity to SEQ ID NO: 23.

6.1.2. Targeting Domain

In some embodiments the BI-1 modulating peptide further comprises a targeting domain capable of transporting the BI-1 modulating peptide across a mammalian cell plasma membrane.

In some embodiments the targeting domain is a cell penetrating peptide. In some embodiments the targeting domain comprises the amino acid sequence set forth in SEQ ID NO: 8. In some embodiments the targeting domain comprises the amino acid sequence set forth in SEQ ID NO: 9. In some embodiments the targeting domain comprises the amino acid sequence set forth in SEQ ID NO: 28. In some embodiments the targeting domain comprises the amino acid sequence set forth in SEQ ID NO: 29. In some embodiments the targeting domain comprises the amino acid sequence set forth in SEQ ID NO: 30. In some embodiments the targeting domain comprises the amino acid sequence set forth in SEQ ID NO: 31. In some embodiments the targeting domain comprises the amino acid sequence set forth in SEQ ID NO: 104. In some embodiments the targeting domain comprises an antibody or a fragment of an antibody.

In some embodiments the targeting domain is at the N-terminus of the BI-1 modulating peptide. In some embodiments the targeting domain is at the C-terminus of the BI-1 modulating peptide.

6.1.3. Chemical Modifications

In certain embodiments the BI-1 modulating peptide comprises at least one chemical modification.

In some embodiments the chemical modification is coupled delivery vehicle. In some embodiments the coupled delivery vehicle is a liposome. In some embodiments the coupled delivery vehicle is a nanoparticle.

In some embodiments the chemical modification is a non-covalent modification. In certain embodiments, the chemical modification is covalently linked. In various embodiments the chemical modification is amidation, acetylation, glycosylation, lipidation, phosphorylation, polyethylene glycol (PEG) modification, or sulfation.

In some embodiments, the chemical modification is a covalent linkage of a fatty acid. In certain embodiments, the fatty acid is saturated. In certain embodiments, the fatty acid is unsaturated.

In some embodiments the chemical modification includes one or more modifications at amino acid side groups, the terminal amine group, or the terminal carboxy group. In some embodiments the chemical modification is a chemical blocking of the terminal amine group. In some embodiments the chemical modification is a chemical blocking of the terminal carboxy group.

6.1.4. Mutations

In certain embodiments, the BI-1 modulating peptide comprises at least one mutation. In some embodiments, the mutation increases the affinity of the BI-1 modulating peptide for binding BI-1. In some embodiments, the mutation decreases the affinity of the BI-1 modulating peptide for binding BI-1. In some embodiments the mutation improves the therapeutic efficacy of the peptide.

In some embodiments, the mutation is an amino acid substitution. In some embodiments, the mutation is an amino acid insertion. In some embodiments, the mutation is an amino acid deletion.

In some embodiments, an original amino acid is substituted by a natural amino acid. In some embodiments, an original amino acid is substituted by an unnatural amino acid. In some embodiments, an original amino acid is substituted by a chemically modified amino acid.

In various embodiments, the amino acid substitution is a conservative or semi-conservative substitution. In some embodiments, the amino acid substitution has minimal impact on the activity and/or structure of the resultant peptide.

In various embodiments, the amino acid substitution is a non-conservative substitution. In some embodiments, the amino acid substitution produces significant changes in the peptide property. In certain embodiments, a hydrophilic residue is substituted by a hydrophobic residue. In certain other embodiments, a hydrophobic residue is substituted by a hydrophilic residue. In certain embodiments, a residue having a bulky side group is substituted by a residue not having a side group. In certain other embodiments, a residue not having a side group is substituted by a residue having a bulky side group.

6.2. Preparation of BI-1 Modulating Peptides

Also disclosed herein are methods for producing the isolated BI-1 modulating peptide.

6.2.1. Recombinant Synthesis

In certain embodiments, the isolated peptide disclosed herein is produced recombinantly, for example using bacterial, yeast, or eukaryotic expression systems.

For recombinant production, a polynucleotide sequence encoding the single or multi-domain peptide is inserted into an appropriate expression vehicle, that is, a vector which contains the necessary elements for the transcription and translation of the inserted coding sequence, or in the case of an RNA viral vector, the necessary elements for replication and translation. The expression vehicle is then transfected into a suitable target cell which will express the single or multi-domain peptide. Depending on the expression system used, the expressed peptide is then isolated by procedures well-established in the art. Methods for recombinant protein and peptide production are well known in the art.

To increase efficiency of production, the polynucleotide can be designed to encode multiple units of the single or multi-domain peptide separated by enzymatic cleavage sites. The resulting polypeptide can be cleaved (e.g., by treatment with the appropriate enzyme) in order to recover the peptide units. This can increase the yield of peptides driven by a single promoter. In some embodiments, a polycistronic polynucleotide can be designed so that a single mRNA is transcribed which encodes multiple peptides, each coding region operatively linked to a cap-independent translation control sequence, for example, an internal ribosome entry site (IRES). When used in appropriate viral expression systems, the translation of each peptide encoded by the mRNA is directed internally in the transcript, for example, by the IRES. Thus, the polycistronic construct directs the transcription of a single, large polycistronic mRNA which, in turn, directs the translation of multiple, individual peptides. This approach eliminates the production and enzymatic processing of polypeptides and can significantly increase yield of peptide driven by a single promoter.

A variety of host-expression vector systems can be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus (CaMV) or tobacco mosaic virus (TMV)) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedron promoter can be used. When cloning in plant cell systems, promoters derived from the genome of plant cells (e.g., heat shock promoters, the promoter for the small subunit of RUBISCO, the promoter for the chlorophyll a/b binding protein) or from plant viruses (e.g., the 35S RNA promoter of CaMV, the coat protein promoter of TMV) can be used. When cloning in mammalian cell systems, promoters derived from the genome of mammalian cells (e.g., metallothionein promoter) or from mammalian viruses (e.g., the adenovirus late promoter, the vaccinia virus 7.5 K promoter) can be used.

6.2.2. Chemical Synthesis

In some embodiments, the isolated peptide of the disclosure is produced by chemical synthesis. In some embodiments, the peptide is produced using liquid phase peptide synthesis techniques. In some other embodiments, the peptide is produced using solid phase peptide synthesis techniques.

Peptides having either the D- or L-configuration can be synthesized by automated solid phase procedures well known in the art. Suitable syntheses can be performed by utilizing “Boc” or “Fmoc” procedures. Techniques and procedures for solid phase synthesis are well-known in the art. The single and multi-domain peptides can also be prepared by way of segment condensation, as described, for example, in Liu et al., Tetrahedron Lett. 37:933-936, 1996; Baca et al., J. Am. Chem. Soc. 117: 1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). This is particularly the case with glycine containing peptides. Other methods useful for synthesizing the single and multi-domain peptides of the disclosure are described in Nakagawa et al., J. Am. Chem. Soc. 107:7087-7092, 1985.

Additional exemplary techniques known to those of ordinary skill in the art of peptide and peptide analog synthesis are taught by Bodanszky, M. and Bodanszky, A., The Practice of Peptide Synthesis, Springer Verlag, New York, 1994; and by Jones, J., Amino Acid and Peptide Synthesis, 2nd ed., Oxford University Press, 2002. The Bodanszky and Jones references detail the parameters and techniques for activating and coupling amino acids and amino acid derivatives. Moreover, the references teach how to select, use and remove various useful functional and protecting groups.

Peptides having either the D- or L-configuration can also be purchased from commercial suppliers of synthetic peptides. Such suppliers include, for example, Advanced ChemTech (Louisville, Ky.), Applied Biosystems (Foster City, Calif.), Bachem (Torrance, Calif.), Anaspec (San Jose, Calif.), and Cell Essentials (Boston, Mass.)

6.2.3. Purification

The peptides or peptide analogs of the disclosure can be purified by many techniques well known in the art, such as reverse phase chromatography, high performance liquid chromatography, ion exchange chromatography, size exclusion chromatography, affinity chromatography, gel electrophoresis, and the like. The actual conditions used to purify a particular single or multi-domain peptide will depend, in part, on synthesis strategy and on factors such as net charge, hydrophobicity, hydrophilicity, and the like, and will be apparent to those of ordinary skill in the art.

In various embodiments, the isolated BI-1 modulating peptide further comprises a purification tag. In some embodiments, the purification tag is a polyhistidine-tag, a myc-tag, or an HA-tag.

6.3. Pharmaceutical Compositions

Also provided herein are pharmaceutical compositions comprising one or more isolated BI-1 modulating peptides described herein, as the active ingredient, and a pharmaceutically acceptable carrier. These compositions comprise, in addition to one or more of the BI-1 modulating peptides, a pharmaceutically acceptable excipient, carrier, buffer, stabilizer, bulking agent, or other excipients well known to those skilled in the art. Such materials should be non-toxic and should not interfere with the efficacy of the active ingredient. The precise nature of the carrier or other materials within the pharmaceutical composition will typically depend on the route of administration, e.g. oral, intravenous, cutaneous or subcutaneous, nasal, intramuscular, intraperitoneal routes. The BI-1 modulating peptide can be formulated, e.g., using any formulation currently used to formulate therapeutic peptides, such as insulins, GLP-1 agonists, and all approved peptides disclosed in the THPdb database of FDA approve therapeutic peptides and proteins (crdd.osdd.net/raghava/thpdb/).

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

6.4. Methods of Treatment

Also provided herein are methods for treating cancer, including, but not limited to breast cancer, brain cancer, cervical cancer, colon cancer, colorectal cancer, lung cancer, ovarian cancer, prostate cancer, rectal cancer, renal cancer, stomach cancer, thyroid cancer, and uterine cancer.

In some embodiments, the methods comprise administering the BI-1 modulating peptide or the pharmaceutical composition as described herein to a subject with cancer. In some embodiments the subject is at risk of developing cancer.

In various embodiments the subject has a solid tumor cancer.

In certain embodiments, the subject is a mammal. In certain embodiments the subject is a human. In some embodiments the subject is an adult. In certain other embodiments the subject is a child.

In various embodiments, the peptide or the pharmaceutical composition is administered in an amount, on a schedule, and for a duration sufficient to reduce tumor growth in the subject. In some embodiments, the peptide is administered in an amount, on a schedule, and for a duration sufficient to decrease tumor volume and/or tumor diameters by 10%, 20%, 25%, 30% or more as compared to levels just prior to initiation of treatment. In certain embodiments, the peptide is administered in an amount, on a dosage schedule, and for a duration sufficient to decrease tumor volume and or/tumor diameter by at least 35%, 40%, 45%, 50% or more. In particular embodiments, the peptide is administered in an amount, on a schedule, and for a time sufficient to decrease tumor volume and/or tumor diameter by at least 55%, 60%, 65%, 70%, 75%, 80%, 85%, 90%, 95% or more.

In some embodiments, the methods comprise administering the BI-1 modulating peptide or the pharmaceutical composition as described herein by intravenous administration. In some embodiments, the methods comprise administering the peptide by subcutaneous injection. In some embodiments, the methods comprise administering the peptide by intrathecal or intra-cisterna magna administration. In some embodiments, the methods comprise administering the peptide by intratumoral injection or peritumoral injection.

In some embodiments, the methods comprise administering the BI-1 modulating peptide in combination with a further treatment, either simultaneously or sequentially dependent upon the condition to be treated. The further treatment may include, but is not limited to a chemotherapeutic agent, a radiation treatment, a small molecule inhibitor, and an antibody or antibody fragment.

Administration of the pharmaceutically useful peptide of the present invention is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc, is within the responsibility of general practitioners and other medical doctors, and typically takes account of the disease or disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

### 7. OTHER EMBODIMENTS

1. A composition comprising: (i) an NleH moiety.
2. The composition of embodiment 1, further comprising: (ii) a targeting moiety coupled to the NleH moiety.
3. The composition according to either one of embodiments 1 or 2, wherein the NleH moiety is, or comprises:
(i) a polypeptide having the sequence disclosed herein as SEQ ID NO.1 or SEQ ID NO.4;
(ii) a biologically active fragment of (i); or
(iii) a biologically active sequence variant of (i) or (ii).
4. The composition according to embodiment 3, wherein the biologically active fragment is a polypeptide having the sequence disclosed herein as SEQ ID NO: 2, SEQ ID NO: 3, SEQ ID NO; 5, or SEQ ID NO: 6.
5. The composition according to either one of embodiments 3 or 4, wherein the biologically active sequence variant has at least 50% sequence identity to SEQ ID NO: 1, SEQ ID NO: 2, SEQ ID NO: 3, SEQ ID NO: 4, SEQ ID NO: 5 or SEQ ID NO: 6.
6. The composition according to any one of embodiments 2 to 5 wherein the targeting moiety binds a tumor-associated antigen.
7. The composition according to embodiment 6 wherein the tumor-associated antigen is human Ephrin-B2, or a homolog thereof.
8. The composition according to either one of embodiments 6 or 7, wherein the targeting moiety is Azurin, or fragment or variant thereof which retains Azurin's characteristic binding activity.
9. The composition according to embodiment 8 wherein the targeting moiety is, or comprises, a polypeptide having the sequence disclosed herein as SEQ ID NO: 8.
10. The composition according to embodiment 9, wherein the composition is a fusion protein with, or comprising, a polypeptide having the sequence disclosed herein as SEQ ID NO: 9.
11. The composition according to embodiment 1 which consists of, or comprises, a polypeptide having the sequence disclosed herein as SEQ ID NO: 10.
12. The composition according to embodiment 6 wherein the targeting moiety is an antibody or an antibody fragment.
13. A binding moiety that competes with NleH (SEQ ID NO: or SEQ ID NO: 4) for binding to BI-1 (SEQ ID NO: 11).
14. A binding moiety that binds the same or overlapping epitope in BI-1 (SEQ ID NO: 11) that is bound by NleH (SEQ ID NO: 1 or SEQ ID NO: 4).
15. The binding moiety according to either one of embodiments 13 or claim 14, wherein the binding moiety binds the polypeptide having the sequence disclosed herein as SEQ ID NO: 12.
16. The binding moiety according to any one of embodiments 13 to 15, wherein the binding moiety binds the polypeptide having the sequence disclosed herein as SEQ ID NO.13.
17. The binding moiety according to any one of embodiments 13 to 16 wherein the binding moiety is an antibody or an antibody fragment.
18. A conjugate comprising the binding moiety according to any one of embodiments 13 to 17 coupled to a functional moiety.
19. The conjugate according to embodiment 18, wherein the functional moiety promotes intracellular internalisation of the conjugate.
20. A nucleotide encoding a composition, binding moiety, or conjugate of any of the preceding claims.
21. A vector comprising the nucleotide according to embodiment 20.
22. A cell transformed with the vector according to embodiment 21.
23. A method of identifying a subject having a proliferative disorder, the method comprising assessing the level of expression or activity of BI-1 in the subject, or in a sample derived from the subject.
24. A method according to embodiment 23 of identifying a subject having a particular risk of developing a proliferative disorder, the method comprising assessing the level of expression or activity of BI-1 in the subject, or in a sample derived from the subject, an increased level of BI-1 expression or activity indicating an increased risk of the subject of developing a proliferative disorder.
25. A method of prognosing a proliferative disorder-related outcome in a subject, the method comprising assessing the activity or expression of BI-1 in the subject, or in a sample derived from the subject.
26. A method according to embodiment 25, wherein an increase in the activity or expression of BI-1 relative to a control sample is indicative of susceptibility to treatment with an agent capable of inhibiting BI-1 activity.
27. A method of selecting patients, preferably human patients, for treatment of a proliferative condition, the method comprising identifying patients having elevated BI-1 activity or expression and selecting thus identified patients for treatment with an agent capable of inhibiting BI-1 activity.
28. A method of selecting patients according to embodiment 27 in which the agent capable of inhibiting BI-1 activity is a composition, binding moiety, or conjugate of any of embodiments 1 to 19.
29. A method according to any one of embodiments 23 to 28, wherein the subject is mammalian.
30. A method according to embodiment 29, wherein the subject is human.
31. A method according to any one of embodiments 23 to 30, wherein the proliferative disorder is cancer.
32. A method according to embodiment 31, wherein the cancer is breast cancer.
33. A method according to any one of embodiments 23 to 32, wherein the level of expression or activity in the subject or sample derived from the subject is determined relative to a control sample.
34. A BI-1 modulator for use in the treatment of a proliferative condition.
35. A BI-1 modulator according to embodiment 34 in which the condition is cancer.
36. A BI-1 modulator according to embodiment 35 in which the cancer is breast cancer.
37. A BI-1 modulator according to any one of embodiments 34-36, wherein the modulator is an inhibitor of BI-1 activity.
38. A method of selecting a pharmaceutical compound useful for the prevention, inhibition or treatment of a proliferative condition, the method comprising providing a group of candidate pharmaceutical compounds for testing, testing the ability of candidate pharmaceutical compounds to bind BI-1 in a test system, and selecting a candidate pharmaceutical compound on the basis of the ability to bind BI-1.
39. A method according to embodiment 38 further comprising the step of determining the cytotoxicity of the candidate chemotherapeutic agent against breast cancer cells in an in vitro and/or in vivo model.
40. A method according to either one of embodiments 38 or 39 in which candidate pharmaceutical compounds which substantially or completely bind BI-1 are selected.
41. A method according to embodiment 40 in which the proportion of BI-1 in the test system which is bound by the candidate is greater than 90%, 80%, 70%, 60%, 50%, 40%, 30%, or 20%.
42. A pharmaceutical composition comprising the composition, binding moiety, conjugate, nucleotide, vector, or BI-1 modulator according to any one of the preceding embodiments and a pharmaceutically acceptable diluent, carrier or excipient.
43. The pharmaceutical composition according to embodiment 42 further comprising a second therapeutic agent.
44. The pharmaceutical composition according to either one of embodiments 42 or 43 for use in a method of treatment.
45. The pharmaceutical composition according to either one of embodiments 42 or 43 for use in a method of treating a proliferative disease.
46. Use of the pharmaceutical composition according to either one of embodiments 42 or 43 in the manufacture of a medicament for use in a method of treating a proliferative disease.
47. A method of treating a subject having a proliferative disease, the method comprising administering to a subject, preferably a human subject, the pharmaceutical composition according to either one of embodiments 42 or 43; optionally wherein treatment of the subject is adjusted according to detected levels of BI-1 activity or expression.
48. The pharmaceutical composition, use, or method, according to any one of embodiments 42 to 47, wherein the proliferative disease is cancer.
49. The pharmaceutical composition, use, or method, according to embodiment 48 wherein the cancer is breast cancer.

### 8. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

Methods

Direct Yeast Two Hybrid (Y2H) Screen

Polynucleotides encoding MQ001 or MQ002 were PCR amplified and cloned downstream of a GAL4 DNA binding domain into pGBT9 (Clontech). The cloned products were expressed in TOP10 (Clontech) and the plasmid purified using the Qiagen Miniprep Kit. Both pGBT9-MQ001 and pGBT9-MQ002 were transformed into Yeast strain AH109 (Clontech) with either empty pGAD424 or that possessing either BI-1, BI-140 (first 40 amino acids of BI-1). pGAD424 BI-1/BI-140 was transformed with empty pGBT9 as a negative control. AH109 was made chemically competent and heat-shocked as described in the Clontech Yeast Protocols Handbook (PT3024-1). The transformed yeast was initially plated on SD minimal agar lacking adenine and histidine, confirming the co-transformation of both pGBT9 and pGADT7. Positive colonies were plated on SD minimal agar plates lacking adenine, histidine, leucine and tyrosine to confirm both the presence of the plasmids and any interaction that occurs between the two cloned proteins of interest.

β-galactosidase Assay

β-galactosidase assays were performed according to the manufacturer's protocols (Clontech PT3024-1 manual). Briefly, pGADT7-BI-1 or pGADT7-BI-140 plasmid alone or with pGBT-MQ001 (or pGBT9, pGBT9-MQ002 when necessary) were transformed into Saccharomyces cerevisiae strain PJ69-4A using the lithium acetate method. Transformants were selected on Trp2 Leu2 plates and grown to an optical density (D600 nm) of 0.6 before lysis and assay for the level of β-galactosidase activity using ONPG as a substrate. Data reported are from at least three biological replicates performed in triplicate.

MTT Cell Viability Assay

Cells were untreated or treated in 24-well tissue culture plates for 24 h before being assessed. The cells were washed once with PBS and replaced with DMEM containing 0.1 mg/ml 3-(4,5-dimethylthiazol-2-yl)-2,5-diphenyltetrazolium bromide (MTT) (Sigma) for 1 h, after which the medium was removed and 100 ml of dimethylsulphoxide (DMSO, Sigma) was added to each well. After thorough mixing on an orbital shaker for 1 min, the absorbance at 540 nm for each well was obtained using a FLUOstar Omega microplate reader (BMG Labtech). Results were obtained from at least three biological replicates performed in triplicate.

Measurement of Cytosolic Calcium (Ca′) Levels

Cytosolic Ca2+ levels were measured using the commercially available fluorescent indicator Fluo-4 Direct (Invitrogen) according to the manufacturer's instructions.

Cells were grown in a 96 well microplate, treated for 12 hours with MQ001, MQ002, Control (CPP-GFP) or positive control (Thapsigargin—˜100 minutes after adding Fluo-4) incubated with Fluo-4 Direct for 1 h at 37° C. Fluorescent intensities were determined using a fluorometer set for excitation at 494 nm and emission at 516 nm.

NBT Assay for Assessing ROS Levels

The reduction of nitro-blue tetrazolium salt (NBT) into a turquoise colored product was used to indirectly estimate the intracellular ROS levels generated in treated MCF-7 and control cells. NBT (100 μl of 1 mg/ml) was added to treated cells, and then incubated for 1 hour in a CO2 chamber at 37° C. The formed crystals were solubilized by the consecutive addition of KOH (120 μl) and DMSO (140 μl). The intensity of the developed color was measured using an ELISA reader at 645 nm. The percentage of reduction of NBT, which is inversely proportional to the ROS generated, was calculated relative to a control treated with ethanol. The results shown are a representative of ROS relative to the untreated control for each cell respective cell line.

Western Blot Analysis

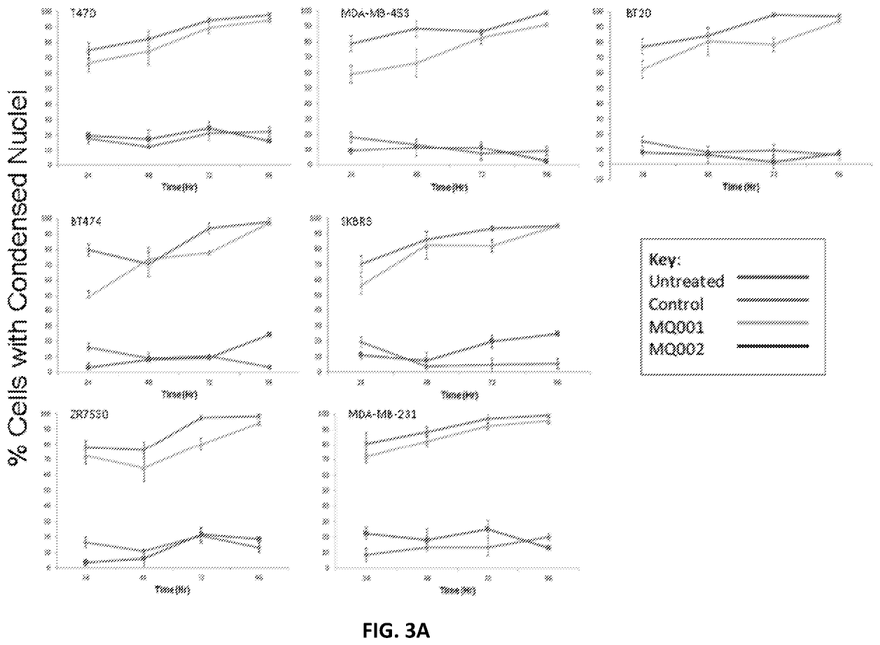

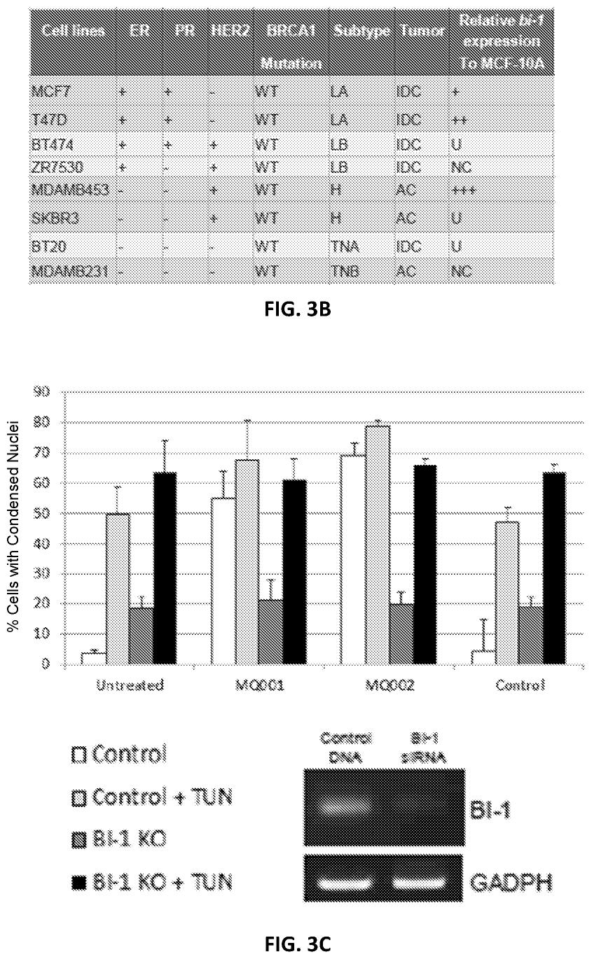

All samples were run on 10-12% SDS-PAGE gels and transferred to a PVDF or Nitrocellulose membrane by conventional methods to compare expression levels for inducible plasmids. The membrane was washed twice with TBS containing 0.1% Tween (Sigma) then incubated for 1 hour with TBS 0.1% Tween containing 5% BSA (Sigma) (for monoclonal antibodies) or 5% commercial powdered milk (polyclonal antibodies). Membranes were incubated for 1 hour (RTP) or overnight (4° C.) with a primary antibody (dilutions used were as indicated by supplier); a list of the antibodies used are provided in Supplementary FIG. 3. The membrane was then washed three times in TBS. 0.1% Tween and incubated with anti-rabbit or anti-mouse secondary antibody (1:2000) conjugated to horse radish peroxidise (HRP) (Cell Signalling) and incubated for 1 hour (RTP). Membranes were developed using ECL reagents (GE Healthcare) and detected in a LAS 3000 Fuji Imager.

Immunoblotting

The following antibodies were used for immunoblotting, anti-His (Sigma), anti-GST (Abcam), anti-tubulin (Abcam), anti-Myc (Abcam), anti-Bcl-2, anti-Bcl-2 (Ser70), anti-Bcl-2 (Thr56), anti-phospho ERK, anti-phospho JNK, anti-ERK, anti-JNK, anti-CHOP, anti-ATF6, Anti-PERK, Anti-IRE1alpha were used for immunoblotting. All blots were performed using 5% BSA in TBS-0.1% Tween. Western blots were stripped using Restore™ Western Blot Stripping Buffer (Thermo Scientific) up to a maximum of four times and probed with different antibodies.

Tissue Culture, MQ001/MQ002 Dosage and Transfection

Cell lines were grown in DMEM containing 1000 mg/L glucose and supplemented with 10% (v/v) fetal calf serum, non-essential amino acids and glutamax in a humidified atmosphere at 5% (v/v) CO2 at 37° C. Cells were treated with MQ001/002 at a final concentration of 0.4 mg/ml, alternatively cells were transfected with either pHM6-BI-1 or pEGFP-N1 (Clontech) (control plasmid) using lipofectamine 2000 (Invitrogen) in accordance with manufacturers protocol and incubated in a humidified atmosphere for 24 h before adding MQ001/002 at a final concentration of 0.4 mg/ml. Cells were then incubated for an additional 24 h. The transfection efficiency for pHM6-BI-1 was ˜30-40%. Control plasmids were transfected at a higher efficiency ˜70%. The difference in transfection efficiency was controlled for during counting, with 100 transfected cells counted in a field of view. siRNA transfection were performed using Hyperfect (Qiagen) in accordance with manufacturers protocol using 20 μM BI-1 or control siRNA. After 72 h, knockdown of BI-1 expression was tested by RNA isolation using the QIAGEN RNAeasy kit according to the manufacturer's recommendation and semi-quantitative RT-PCR using BI-1 (h)-PR (Santa cruz, sc-37298-PR) or GADPH primers.

Immunofluorescence Staining and Co-Localization

Semi-confluent cell monolayers were grown on coverslips and fixed with 3% paraformaldehyde (Sigma) in phosphate buffered saline (PBS) pH 7.4 for 15 minutes at RTP. The cells were washed 3 times with PBS and the paraformaldehyde neutralized with ammonium chloride (10 mM) for 10 minutes. Cells were permeabilized with PBS containing 0.2% Triton-X (Sigma) for 4 minutes and washed twice more in PBS, then incubated for 10 minutes in PBS containing 1% bovine serum albumin (BSA) (Sigma). Cells were then incubated with primary antibodies for 1 hour at RTP, or overnight at 4° C. if recommended by manufacturer's guidelines. Coverslips were washed twice in PBS and once more with PBS containing 1% BSA, they were incubated with the appropriate secondary antibody for 45 minutes. In the instance of co-localization the primary antibody and secondary corresponding to that primary was put on first. The second primary was added and it was ensured the secondary antibody was from a different source (i.e. if first primary antibody was raised in mice, the second primary antibody would be raised in rabbit). Dyes such as MitoTracker or DAPI were used in accordance with manufacturers guidance.

Coverslips were washed three times in PBS and once more in autoclaved distilled water and mounted on slides using ProLong Gold antifade (Invitrogen). Coverslips were visualised on a Zeiss Axioimager immunofluorescence microscope at 32 or 100 times magnification, and analysed using Axiovision Rel 4.5 software. Cells were counted within numerous fields of vision, counting at least 600-9000 cells from any one coverslips; all experiments were repeated three to five times. All counts were performed in a double-blind manner. All antibodies came from Cell Signaling unless otherwise stated. Anti-rabbit IgG, HRP-linked Antibody #7074, Anti-mouse IgG, HRP-linked Antibody #7076, Anti-BI-1 antibody (ab18852)(Abcam), GST Antibody #2622, Anti-GST antibody (ab19256)(Abcam), His-Tag (27E8) Mouse mAb #2366, HA-Tag (6E2) Mouse mAb #2367, Anti-Myc tag antibody (ab9106)(Abcam), β-Actin (8H10D10) Mouse mAb #3700, MitoTracker® Red CMXRos #9082, Anti-Tubulin antibody—Loading Control (ab59680)(Abcam), α/β-Tubulin Antibody #2148, Calnexin Antibody #2433, LC3A/B (D3U4C) XP® Rabbit mAb (Alexa Fluor® 594 Conjugate) #14079, Phospho-Bcl-2 (Ser70) (5H2) Rabbit mAb #2827, Phospho-Bcl-2 (Thr56) Antibody (Human Specific) #2875, Bcl-2 (D55G8) Rabbit mAb (Human Specific) #4223, p44/42 MAPK (Erk1/2) (137F5) Rabbit mAb #4695, Phospho-SAPK/JNK (Thr183/Tyr185) Antibody #9251, Phospho-p44/42 MAPK (Erk1/2) (Thr202/Tyr204) (197G2) Rabbit mAb #4377.

Flow Cytometry and Cell Sorting

Cells were grown in a T75 flask until ˜70% confluence (1.6 e106 cells) and treat at a concentration of 0.2 mg/ml of MQ001 or control for a 96-hour period. Every 24 hours a sample was taken, and all cells assessed based on their forward and side scatter patterns. The forward scatter correlates with the cells volume, while the side scatter correlates with the inner complexity of the cell (i.e. shape of nucleus, amount of cytoplasmic granules or membrane roughness). Adherent cells on the flask were washed in PBS and trypsanized then resuspended in DMEM to generate a single cell suspension. The cell suspension was immediately assessed on the BD LSRFortessa (BD Biosciences). MQ001 and the control both had a GFP tag, in the MQ001 sample to show uptake and treatment of cells with MQ001 internalized these are highlighted in black.

Annexin V and Condensed Nuclei Counts

Cells were grown in Dulbecco's modified Eagle's medium low glucose (1 g/liter; Invitrogen) supplemented with 10% fetal bovine serum, non-essential amino acids (Sigma) to 70% confluence in a T25 flask. Cells were then treated with MQ001, MQ002, Control (CPP-GFP) or untreated 12 h to induce apoptosis. Both floating and attached cells were then collected and labeled using the annexin V-fluorescein isothiocyanate apoptosis detection kit (catalog number K101-100; BioVision), following a protocol provided by the manufacturer. 104 cells from each condition were analyzed by FACS to identify cells into that were Annexin V-positive. Aliquots of the collected cells were also suspended at 106 cells/ml in PBS with 2 g/ml Hoechst dye, and the percentage of cells with apoptotic nuclear morphology was determined by UV microscopy. This data was confirmed by individual counts and repeats of the experiment using immunofluorescence as described above.

RT-PCR

Verso one-step RT-PCR (Thermo Scientific) was performed in accordance with manufacturer's protocol. Primers were purchased from Sigma as part of an Apoptosis multiplex kit.

Immunoprecipitation/Co-Immunoprecipitation

Cells were grown as stated in T75 cm² flasks and were transfected with pHM6-MQ001 or pHM6-MQ002 for 24 hours prior to lysis in IP buffer (50 mM Tris-HCl pH7.4, 150 mM NaCl, 1% Triton X-100, 1 mM EDTA, 2 mM Sodium Orthovanadate, 10 mM Sodium fluoride, 1 mM PMSF, EDTA-free Protease cocktail inhibitor). Endogenous BI-1 was immunoprecipitated, using Anti-HA Magnetic Beads (Pierce) to capture the HA-tagged MQ001/002. Beads were washed three times with IP buffer and finally resuspended in 200 □l of IP buffer. 20 μl of samples was loaded with 5 μl SDS loading buffer. All samples were boiled for 5 min, subjected to SDS-PAGE and transferred to nitrocellulose membranes. Membranes were probed with the following primary antibodies as necessary: Anti-BI-1 (Calbiochem), anti-tubulin (Cell Signaling) and anti-HA (Sigma) antibodies were used for immunoblotting. All antibodies were diluted in accordance with manufacturer's instructions and left overnight TBS-Tween (0.1%) with 5% BSA. Images were visualized using an MFChemiBis imaging station (DNR).

Microsomal Stability

The viability of the microsomal reaction system was confirmed by loss of 7-ethoxycoumarin (m/z 191) and formation of 7-hydroxycoumarin (7-OHC, m/z 163) and 7-OHC glucuronide (m/z 339). Microsomes were incubated with uridine 5′-diphospho-glurcuronic acid (UDPGA) cofactor solution A (BD Gentest) at a reaction concentration of 2 along with solution B (BD Gentest); 50 mM Tris-HCl, 8 mM MgCl2, and 25 alamethicin in deionized water. MQ001 was added to individual reaction mixtures to yield a final concentration of 10 μM and reaction mixtures (0.5 ml) incubated in triplicate at 37 C for the defined time and quenched with 0.1 ml 7% perchloric acid, centrifuged at 12,500 rpm (11,0009 g) for 5 min. Supernatants were transferred to autosampler vials for analysis. The reaction system was validated using a substrate/metabolite-positive control (7-ethoxycoumarin/7-hydroxycoumarin; 7-ethoxycoumarin/7-hydroxycoumarin glucuronide) and four negative control reactions conducted in parallel with each set of substrate reactions.

Animal Experiments

All mice were handled in accordance with the 1986 Animal Scientific Procedures Act and experimentation was carried out under a United Kingdom Government Home Office-approved project license 70/8586. On day 0 all animals received MCF-7 or MDA-MB-231 human breast cancer cells (ATCC) were cultured in aMEM containing 5% FBS (Life Technologies). Both cell lines were grown in T-150 flasks and yielded 5-10×106 cells/flask depending on confluence. For inoculation into balb/c mice, cells were washed with PBS, trypsinized, centrifuged and resuspended in 0.3 ml Matrigel-αMEM.

Female BALB/c mice (n=30 per breast cancer type; 8 weeks old; 18-20 g) were randomly assigned to each group (n=10); the three groups were PBS, control, MQ001. Animals were housed at 22±5° C. in a 12 h light/dark cycle and fed rodent chow and water freely. Orthotopic mammary fat pad implantation was performed as follows: Female BALB/c mice were inoculated with the aforementioned cell resuspension in the mammary fat pads under anesthesia via Matrx VMS anesthesia machine (Midmark Corporation) by continuous inhalation of 2% isoflurane gas for 5-10 min. Sterile tweezers were used to lift the fourth nipple and a syringe needle (BD Biosciences) was used to implant cell or tissue suspensions directly into the mammary fat pad. In all studies, mice were implanted sc with 17β-estradiol-sustained release pellets (Innovative Research). The tumor take rate ranged from 95-100%.

Tumor length (L) and width (W) were measured twice weekly using calipers, and tumor volume (V) was calculated as [V=(L×W2)/2]. After 3 weeks, mice bearing tumors with volumes averaging approximately 200 mm3 were utilized for treatment. In the studies, tumor-bearing mice were treated daily for 5 days with PBS, Control or MQ001 at a dose of 10 mg/kg (3.4 μmol/kg). Tumor volume was determined three times a week. Body weights were measured twice weekly. Mice were sacrificed on day 20 due to the significant reduction in tumour size and volume of MQ001 treated mice. Significance (P<0.001) between control groups and MQ001 treated groups was determined using a series of mixed-model analyses as described in Statistical Methods. A log-quadratic mixed-model fit the data and identified 10 mg/kg MQ001 as significantly different than control or PBS.

Histopathology

Segments of the lung, heart, brain, kidney, spleen and liver each mouse were collected at the final end point (20 days after treatment) rinsed of their content and fixed in 10% buffered formalin for microscopic examination. Formalin-fixed tissues were then processed, paraffin embedded, sectioned at 5 μm, and stained with hematoxylin and eosin (H&E) according to standard techniques.

### Example 1: Peptide Design

BI-1 modulating peptides were designed to interact with and modulate the cellular regulator BI-1. BI-1 can signal cellular pathways to inhibit, delay, or promote apoptosis as well as cell survival by adapting to pro-apoptotic and anti-apoptotic stimuli (Robinson et al., Oncogene 30: 2391-2400, 2011). The NIeH family of bacterial protein effectors have been shown to bind to BI-1 and inhibit apoptotic signaling (Hemrajani et al., Proc Natl Acad Sci 107: 3129-3134, 2010).

In order to make a therapeutically useful peptide that would interact with and modulate BI-1 in cancer cells, fusion proteins were made with a 28 amino acid domain of the Pseudomonas protein azurin (p28) and the NIeH effector protein NIeH1. The p28 domain has been shown to be responsible for azurin's preferential entry into cancer cells (Yamada et al., Mol. Cancer Ther. 8: 2947-2958, 2009).

The BI-1 modulating peptide MQ001 was created by cloning polynucleotides encoding p28 and NIeH1 into a bacterial expression vector in a single reading frame with the nucleotide sequence encoding p28 5′ to the nucleotide sequence encoding NIeH1. The resulting plasmid DNA was amplified and transformed into E. coli. Cultures of E. coli transformed with the plasmid DNA were subsequently grown, harvested, and purified using the methods provided above to isolate the p28-NIeH1 fusion protein. The p28-NIeH1 fusion protein MQ001 has the amino acid sequence set forth in SEQ ID NO: 16.

Additional BI-1 modulating peptides were designed based on structural algorithms to ensure minimal interference between the therapeutic peptide, BI-1, and other potentially interacting proteins. Several BI-1 modulating peptides were created by modifying the C-terminal sequence of NIeH1. The BI-1 modulating peptide MQ157 (SEQ ID NO: 17) was generated using the 157 C-terminal amino acids of NIeH1. The BI-1 modulating peptide MQ70 was generated using the 77 C-terminal amino acids of NIeH modified by the addition of alanine, serine, and methionine at the N-terminus of the peptide (SEQ ID NO: 18). Additional BI-1 modulating peptides MQ30 (SEQ ID NO: 19), MQ22 (SEQ ID NO: 20), MQ16 (SEQ ID NO: 21), MQ8A (SEQ ID NO: 22), MQ8B (SEQ ID NO: 23), MQ45 (SEQ ID NO: 24) and MQ60 (SEQ ID NO: 25) all have amino acid sequences that align with a portion of the C-terminus of NIeH1.

Further BI-1 modulating peptides were created by the addition of peptide sequences ranging from 9 to 28 amino acids on either the N- or C-terminal end of the previously generated therapeutic peptides. The additional peptide sequences confer cancer cell targeting and cell membrane penetrating properties on the BI-1 modulating peptides. Sequences of these additional BI-1 modulating peptides are shown in the sequence listing table in Section 10 below (SEQ ID NOs 32-103).

### Example 2: Exemplary BI-1 Modulating Peptides Interact with the Amino Terminus of BI-1

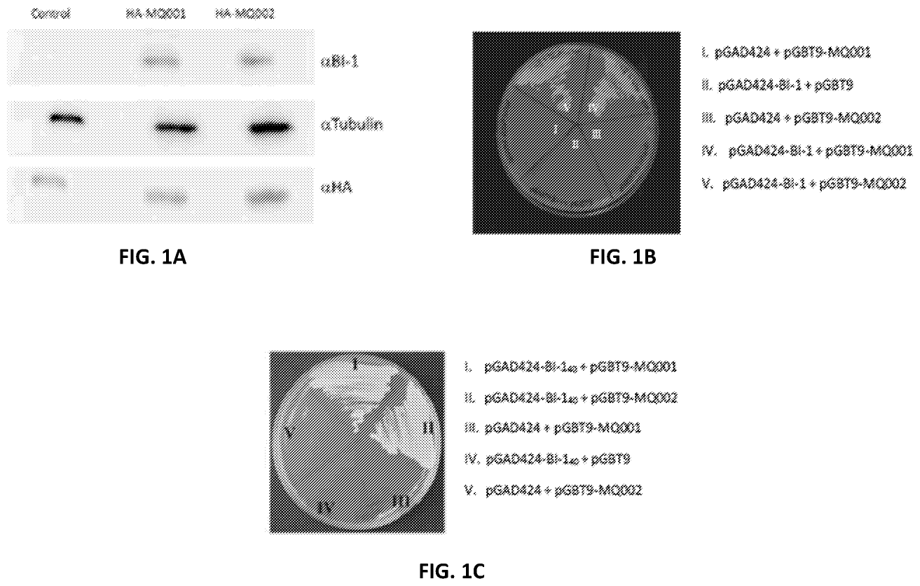

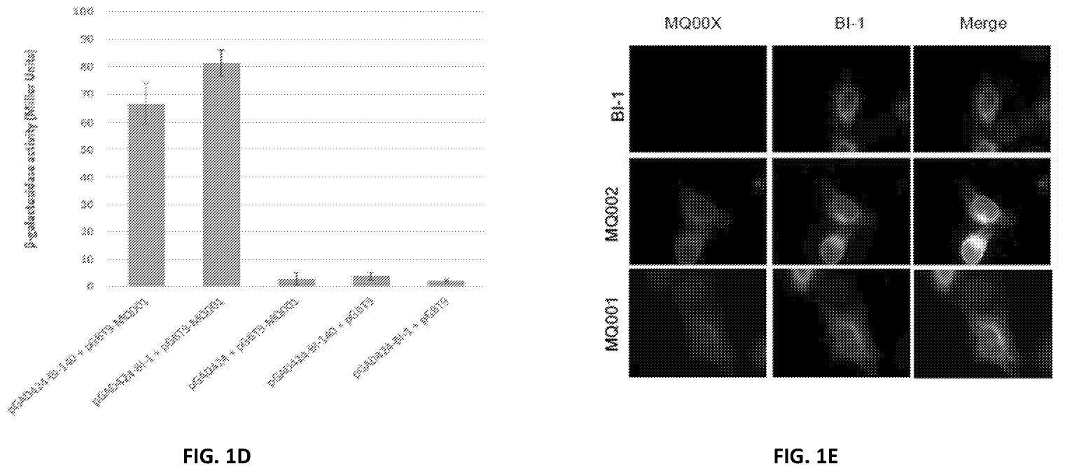

The BI-1 modulating peptides MQ001 and MQ002 were assessed for the ability to directly interact with BI-1. Results of an immunoprecipitation of lysates from HeLa cells transfected with HA-tagged MQ001, MQ002, or GFP (control) show that MQ001 and MQ002 both interact with BI-1 as endogenous BI-1 co-immunoprecipitated with the HA-tagged peptides following incubation with anti-HA magnetic beads (FIG. 1A).

Direct yeast two-hybrid screens were performed in Saccharomyces cerevisiae to confirm the interaction between both BI-1 modulating peptides MQ001, MQ002 and BI-1. Results demonstrated that both MQ001 and MQ002 separately interact with BI-1 (FIG. 1B). Additional yeast two-hybrid screens further demonstrated that both MQ001 and MQ002 separately interact with at least a portion of the 40 N-terminal amino acids of BI-1 (FIG. 1C).

β-galactosidase reporter assays were also performed using S. cerevisiae in order to further confirm that MQ001 interacts with BI-1. The level of β-galactosidase activity measured in a given assay can be used to compare the relative strength of the protein-protein interactions of selected transformants. Results shown in FIG. 1D demonstrate that the strength of the interaction between MQ001 and the N-terminus of BI-1 is only slightly reduced compared to the interaction of MQ001 and full-length BI-1.

HeLa cells co-transfected with either HA-tagged MQ001 or MQ002 and Myc-tagged BI-1 were fixed and incubated with fluorophore-conjugated anti-HA and anti-Myc antibodies. Immunofluorescence images of the treated cells show MQ001 and MQ002 each co-localize with BI-1 (FIG. 1E).

### Example 3: Exemplary BI-1 Modulating Peptides Induce Cell Death in Breast Cancer

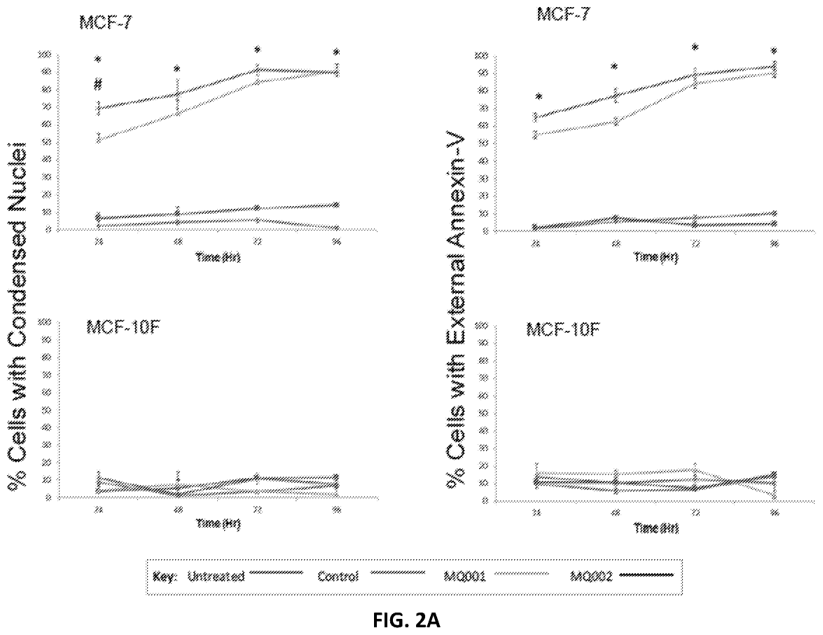

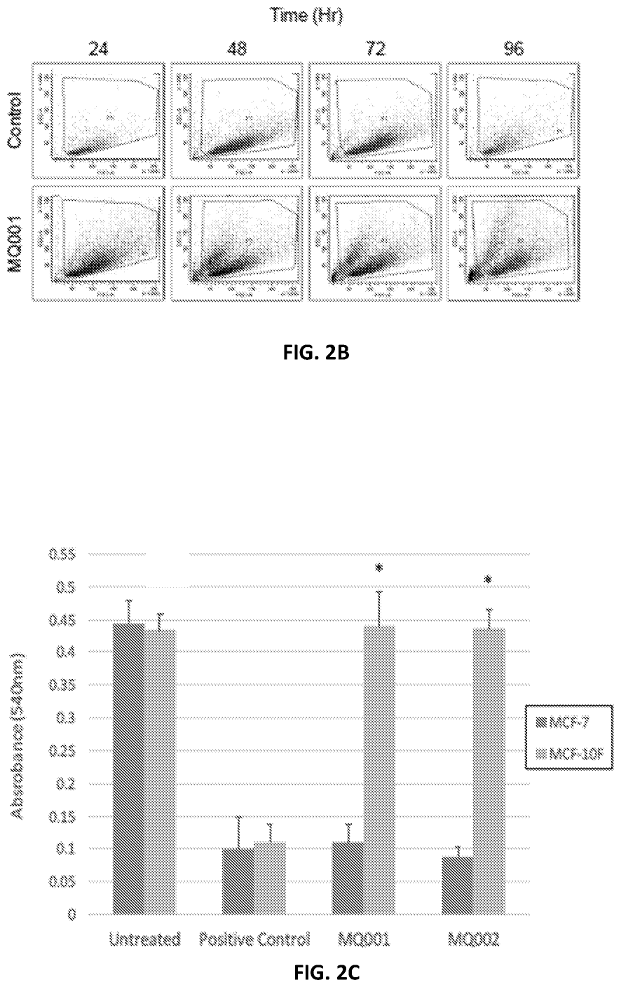

Cells derived from cancerous breast tissue (MCF-7) and non-cancerous breast tissue (MCF-10F) were separately treated with MQ001, MQ002, or GFP (control) for 12 hours and then assessed for markers of apoptosis, including condensed nuclei and the presence of annexin-V on the cell surface using the methods described above. Results (FIG. 2A) show that MQ001 and MQ002 both induce apoptosis in breast cancer cells but do not induce apoptosis in breast cells derived from healthy breast tissue.

In order to further evaluate viability of both breast cancer and non-cancerous breast cells following treatment with MQ001 or MQ002, cells were treated with either MQ001, MQ002, or left untreated (control) for 24 hours and then assessed by MTT assay. Results in FIG. 2C show that MCF-7 cells treated with either MQ001 or MQ002 showed significantly lower levels of the reduced form of MTT, suggesting that treatment with either MQ001 or MQ002 reduced the number of viable MCF-7 cells while leaving MCF-10F cells relatively unaffected.

MCF-7 cells were subsequently treated for 96 hours with GFP-tagged MQ001 or GFP only (control). Cell samples were assessed by flow cytometry every 24 hours for their forward and side scatter patterns. Considerably more forward and side scatter were seen in cells treated with MQ001 compared to control, indicative of a larger population of dying cells in the MQ001 treated cells (FIG. 2B).

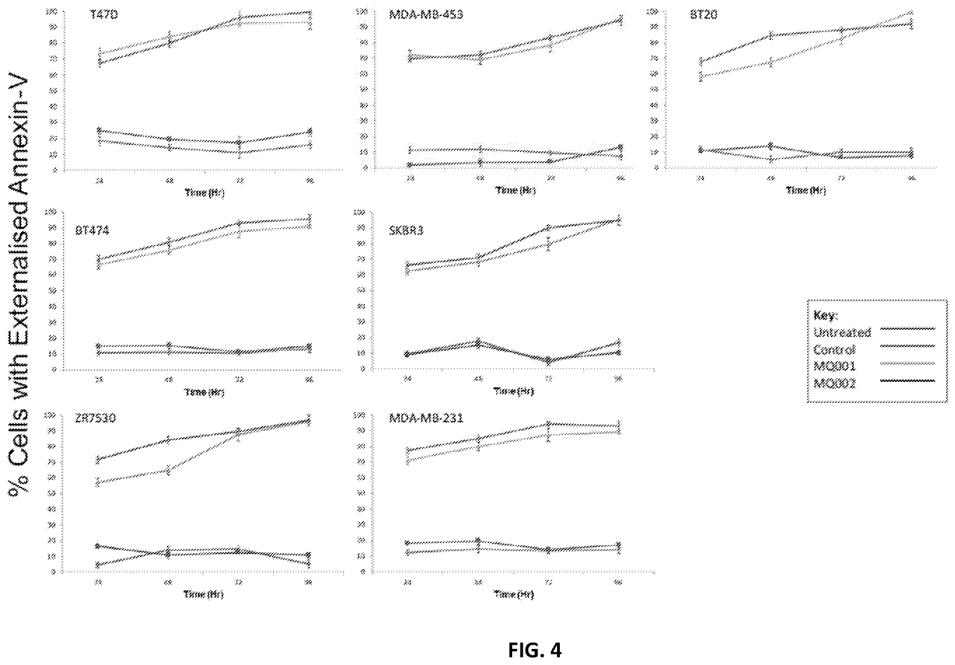

In order to assess whether MQ001 and MQ002 induce cell death in all breast cancer derived cell lines or only MCF-7 cells, seven additional breast cancer derived cell lines were treated with MQ001, MQ002, or GFP (control) and then assessed for condensed nuclei and presence of external annexin-V, as described above. Results shown in FIG. 3A and FIG. 4 demonstrate that treatment with either MQ001 or MQ002 induced 100% cell death in all seven breast cancer cell lines within a 96 hour period. The seven breast cancer lines evaluated were from five breast cancer subtypes, as identified in FIG. 3B.

In order to evaluate the importance of BI-1 on the ability of MQ001 and MQ002 to induce cell death in MCF-7 cells, BI-1 antisense oligonucleotide was used to knock down BI-1 expression (BI-10. Results in FIG. 3C show that treatment with MQ001 or MQ002 had no effect on MCF-7 cells transfected with BI-1_{kd}, indicating that BI-1 is important for the therapeutic effects of MQ001 and MQ002 on breast cancer cells.

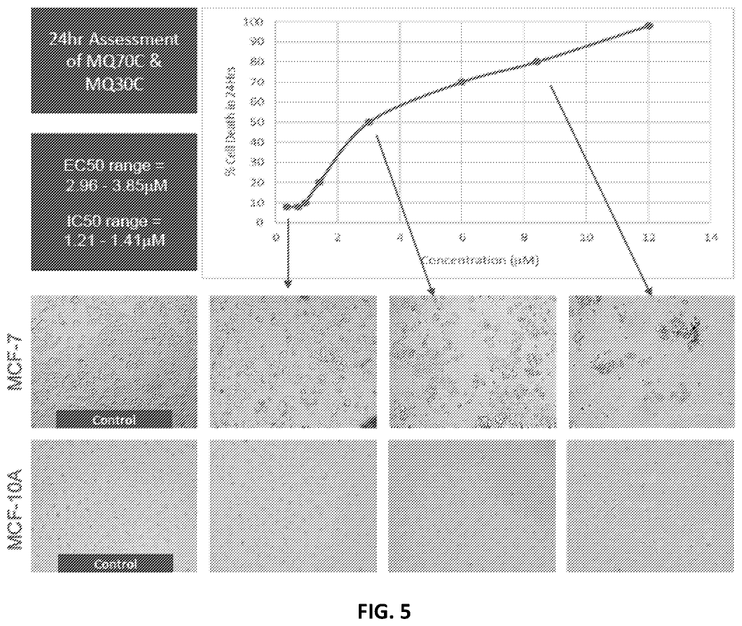

Additional studies were performed to assess the effect of treatment with the BI-modulating peptides MQ70C and MQ30C on cells derived from breast cancer tissue (MCF-7) and cells derived from non-cancerous breast tissue (MCF-10A). Cells were treated for 24 hours with various concentrations of either MQ70C or MQ30C. Quantified results shown in FIG. 5 demonstrate that the BI-1 modulating peptides MQ30C and MQ70C induce MCF-7 cell death in a dose-dependent manner.

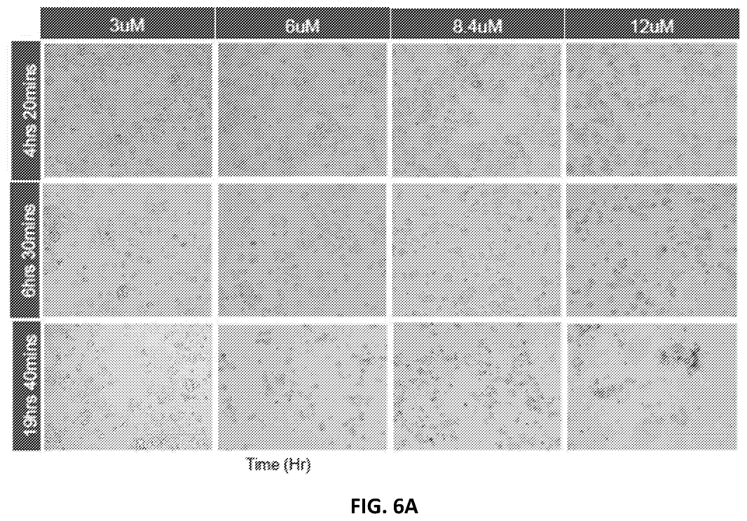

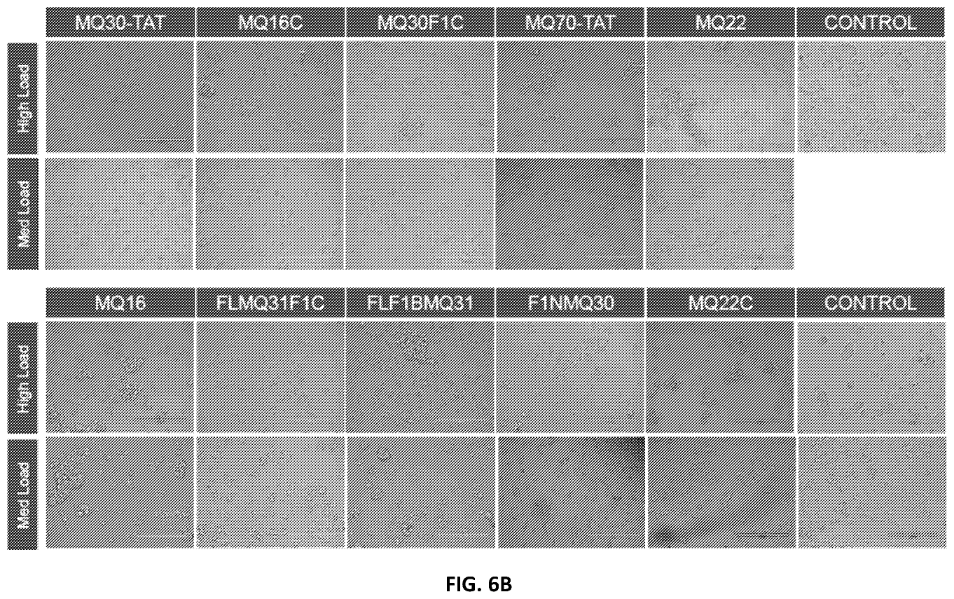

MCF-7 cells treated with 3 μM, 6 μM, 8.4 μM, and 12 μM MQ70C were imaged by phase contrast microscopy following treatment for 4 hours 20 minutes, 6 hours 30 minutes or 19 hours 40 minutes. Morphologic changes in the cells consistent with cell death are visible within a shorter period of time in cells treated with a higher concentration of MQ70C (FIG. 6A) compared to cells treated with a lower concentration of MQ70C, consistent with the assessment that BI-1 modulating peptides induce cell death in breast cancer cells in a dose-dependent manner.

In order to evaluate multiple BI-1 modulating peptides and compare the ability of each peptide to induce cell death, MCF-7 cells were separately treated for 6.5 hours with each of the following BI-1 modulating peptides: MQ30-TAT (SEQ ID NO: 105), MQ16C (SEQ ID NO: 71), MQ30F1C (SEQ ID NO: 49), MQ70-TAT (SEQ ID NO: 106), MQ22 (SEQ ID NO: 20), MQ16 (SEQ ID NO: 21), FLMQ31F1C (SEQ ID NO: 57), FLF1BMQ31 (SEQ ID NO: 56), F1NMQ30 (SEQ ID NO: 48), and MQ22C (SEQ ID NO: 63). MCF-7 cells were treated with each BI-1 modulating peptide at a high load (1 mg/mL peptide concentration) and a medium load (0.6 mg/mL peptide concentration). Results are shown in FIG. 6B.

### Example 4: Exemplary BI-1 Modulating Peptides Induce Cell Death in Multiple Cancer Types

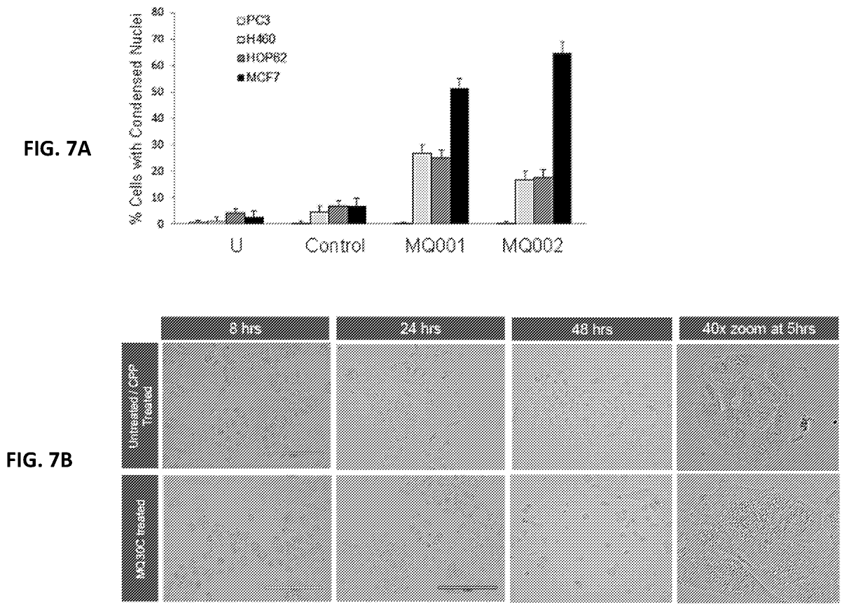

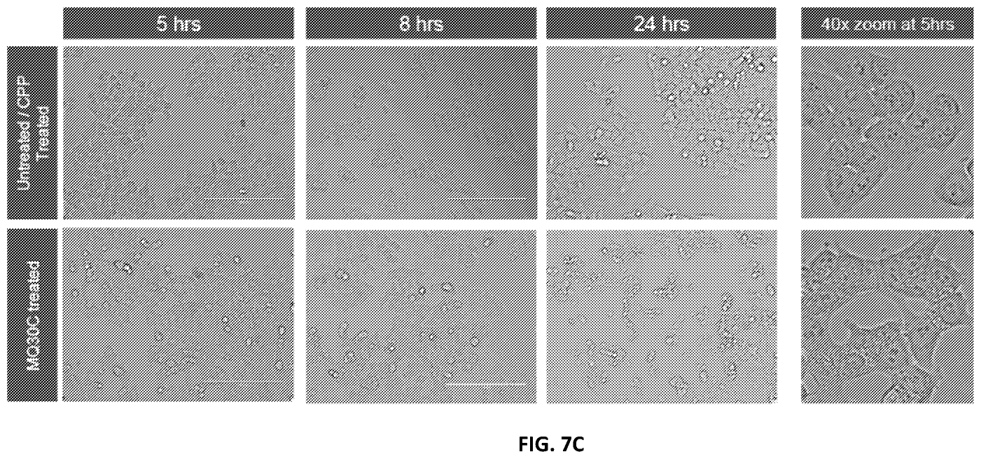

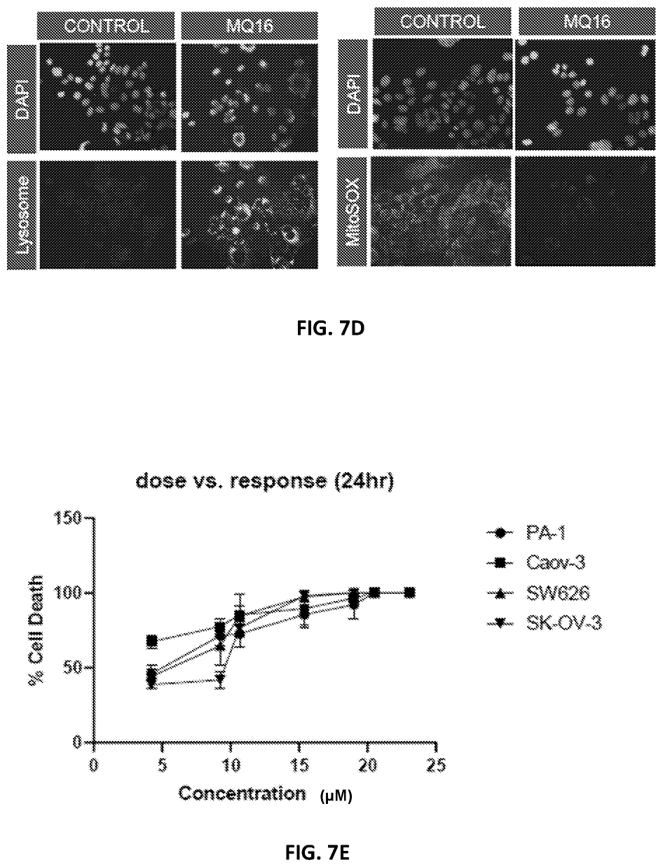

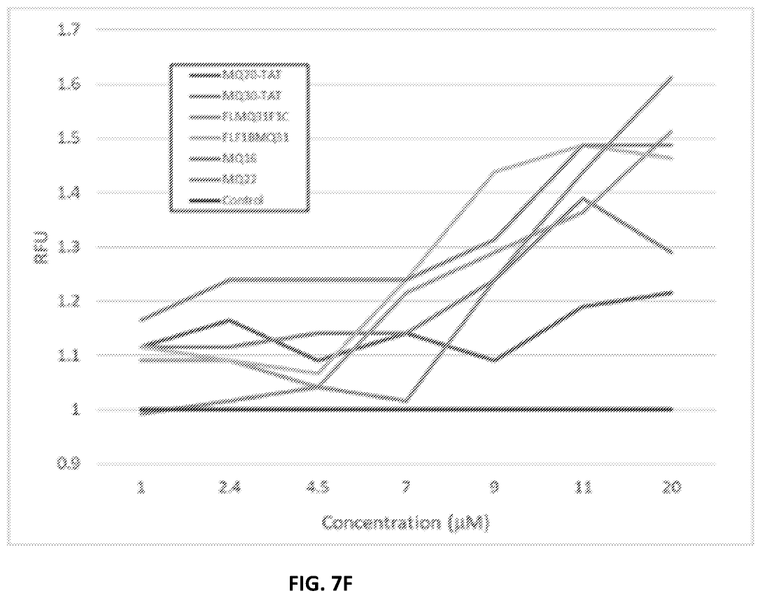

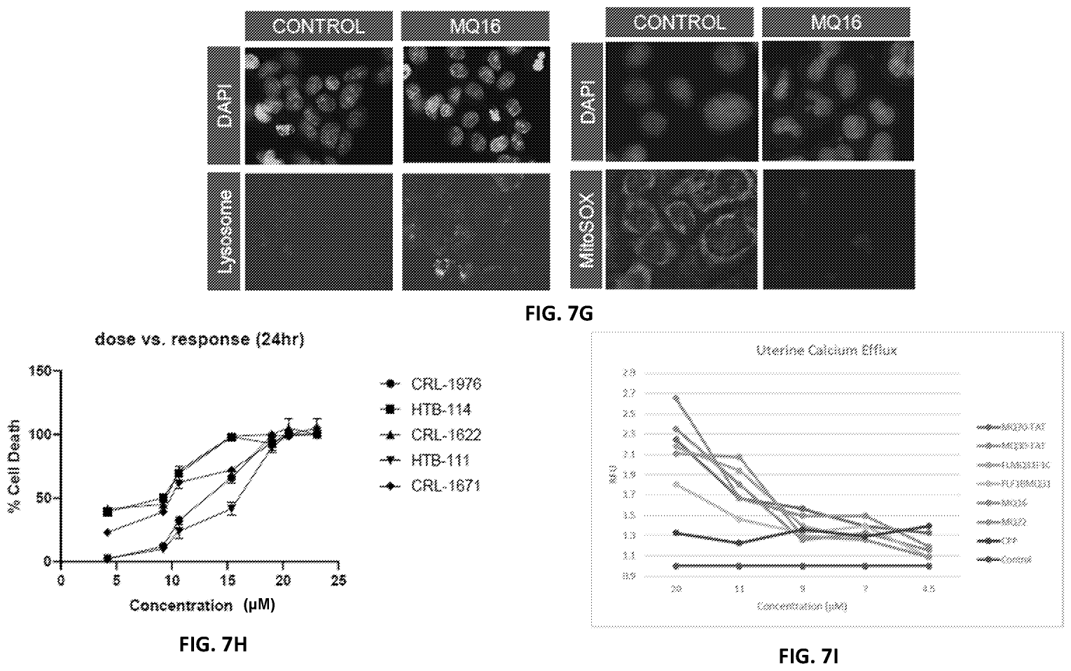

In order to determine whether BI-1 modulating peptides induce cell death in cancer cells other than breast cancer, cell lines derived from cancer tissue other than breast cancer were treated with BI-1 modulating peptides and assessed for markers of cell death. The effects of treatment with MQ001 and MQ002 were assessed in the lung cancer cell lines HOP64 and H460 as well as the prostate cancer cell line PC3. Results in FIG. 7A show that MQ001 induces cell death in approximately 30% of lung cancer cells and MQ002 induces cell death in approximately 20% of lung cancer cells, compared to approximately 50% and 60% induction of cell death in MCF-7 breast cancer cells. In contrast, treatment of prostate cancer cells with MQ001 and MQ002 did not induce cell death (FIG. 7A).

Lung cancer cells (A549) and colon cancer cells (HCT-116) were treated with 0.14 mg/mL of the BI-modulating peptide MQ30C. Cells were imaged by phase contrast microscopy at multiple time points up to 48 hours (lung cancer cells) or 24 hours (colon cancer cells). Results in FIG. 7B and FIG. 7C show that the morphology of most of the cells treated with MQ30C change over time. 40× images taken following 5 hours of treatment appear to show disruption of the ER in cells treated with MQ30C compared to cells treated with CPP alone. Greater than 85% of the A549 and HCT-116 cells treated with MQ30C were dead following 96 hours of treatment (data not shown).

The ovarian cancer panel (ATCC-1021) was challenged with BI-modulating peptides and cells were assessed by phase-contrast and immunofluorescence microscopy for morphological changes including nuclear condensation, ER disruption, lysosome formation, and mitochondrial membrane permeabilization. Cells were also assessed for changes in cytosolic calcium levels, reactive oxygen species (ROS) levels, and by trypan blue and cell viability assays. Immunofluorescence images of cells from the ovarian cancer cell line SW626 (FIG. 7D) show formation of lysosomes and permeabilization of mitochondrial membranes in cells treated with the BI-1 modulating peptide MQ16 compared to control, consistent with cell death induced by MQ16. Dose response curves of MQ16 treatment in each of four ovarian cancer cell lines are shown in FIG. 7E. Cytosolic calcium levels were measured in SW626 cells following treatment with various BI-1 modulating peptides. Results in FIG. 7F show calcium efflux in the cells in response to treatment with each BI-1 modulating peptide but not in response to treatment with CPP-GFP (control), suggesting that the therapeutic peptides induce release of calcium from intracellular calcium stores thereby promoting cell death.

The uterine cancer panel (ATCC-1023) was challenged with BI-modulating peptides and cells were assessed by phase-contrast and immunofluorescence microscopy for morphological changes including nuclear condensation, ER disruption, lysosome formation, and mitochondrial membrane permeabilization. Cells were also assessed for changes in cytosolic calcium levels, reactive oxygen species (ROS) levels, and by trypan blue and cell viability assays. Immunofluorescence images of cells from the uterine cancer cell line CRL-1671 (FIG. 7G) show formation of lysosomes and permeabilization of mitochondrial membranes in cells treated with the BI-1 modulating peptide MQ16 compared to control, consistent with cell death induced by MQ16. Dose response curves of MQ16 treatment in each of five uterine cancer cell lines are shown in FIG. 7H. Cytosolic calcium levels were measured in CRL-1671 cells following treatment with various BI-1 modulating peptides. Results in FIG. 7I show calcium efflux in the cells in response to treatment with each BI-1 modulating peptide but not in response to treatment with CPP-GFP (control), suggesting that the therapeutic peptides induce release of calcium from intracellular calcium stores thereby promoting cell death.

### Example 5: Exemplary BI-1 Modulating Peptides do not Exhibit Negative Effects on Non-Cancerous, Non-Stress Induced Cells

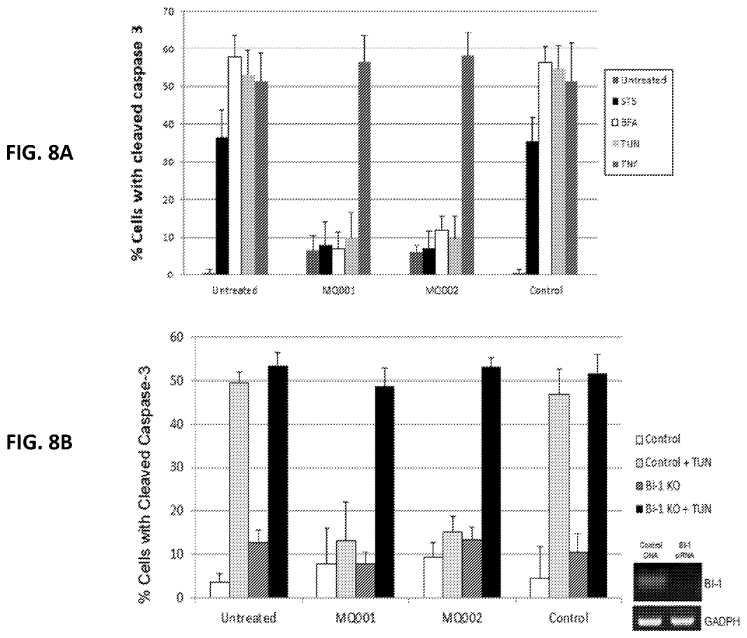

To evaluate whether MQ001 and MQ002 induced anti-apoptotic effects in non-cancerous cells, MCF-10F cells were treated with MQ001 or MQ002 for 24 hours and then exposed to an intrinsic inducer of apoptosis: staurosporine (STS), tunicamycin (TUN), or Brefeldin A (BFA) or extrinsic inducer of apoptosis: TNFα (TNF). Results in FIG. 8A show that that MQ001 and MQ002 both prevent apoptosis in MCF-7 cells subjected to intrinsic inducers of apoptosis but have no anti-apoptotic effect on MCF-7 cells subjected to the extrinsic inducer of apoptosis, TNF. In combination with the results of experiments outlined in Examples 3 and 4, this data suggests that BI-1's pro- and anti-apoptotic functions can both be modulated by BI-1 interacting peptides, with BI-1 alternatively functioning to induce or prevent cell death depending on the nature of the cell.

To further investigate the importance of BI-1, BI-1 antisense (BI-1_{kd}) was used to knock down BI-1 expression in MCF-7 and MCF-10F cells that were then treated for 24 hours with MQ001, MQ002, control (GFP-CPP), or were left untreated. Cells were subsequently exposed to stress inducing agents and apoptosis inducers TUN, BFA, or STS. Results in FIG. 8B show that MCF-10F cells lacking BI-1 and that are exposed to the stress inducing agent TUN are induced to undergo apoptosis, irrespective of MQ001 or MQ002 treatment. This data further establishes that BI-1 is necessary for both the pro- and anti-apoptotic responses induced by MQ001 and MQ002 with the type of therapeutic response depending on the nature of the cell line being treated.

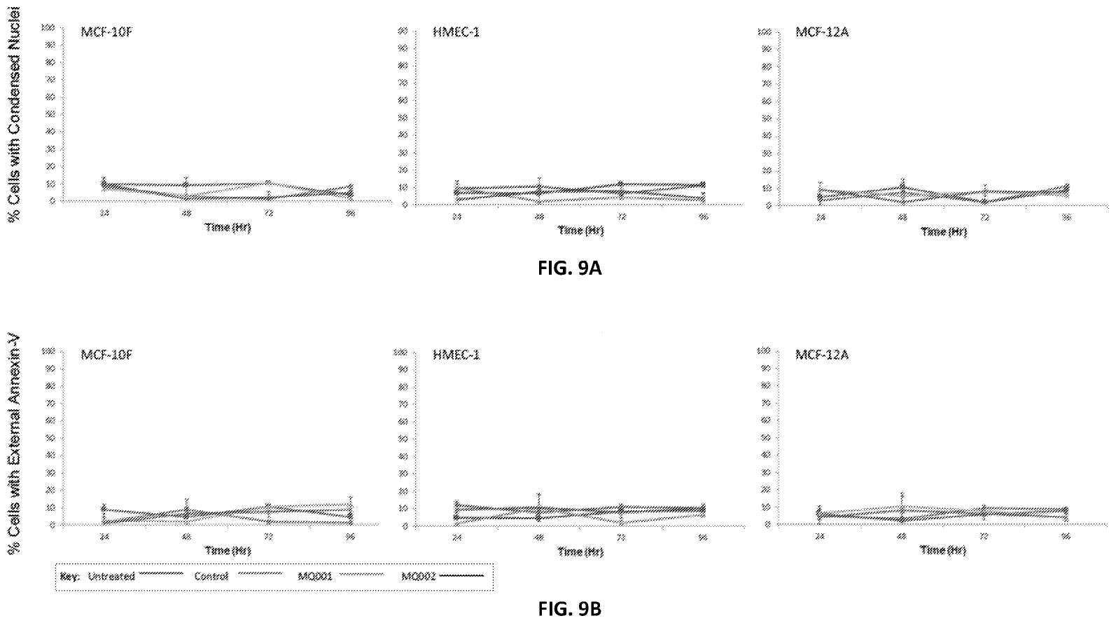

Additional studies were performed on the non-cancerous human cell lines MCF-10F, HMEC-1, and MCF-12A to assess markers of apoptosis following treatment with MQ001, MQ002, or control (GFP-CPP) as previously described. Results in FIGS. 9A and 9B show that treatment with MQ001 and MQ002 did not induce cell death in the non-cancerous cell lines MCF-10F, HMEC-1, and MCF-12A. This data, in combination with that obtained from experiments outlined in Examples 3 and 4, indicates that the BI-1 modulating peptides MQ001 and MQ002 selectively induce cell death in cancer cells.

### Example 6: Treatment with Exemplary BI-1 Modulating Peptides Specifically Elevates Cytosolic Calcium Levels in Cancer Cells

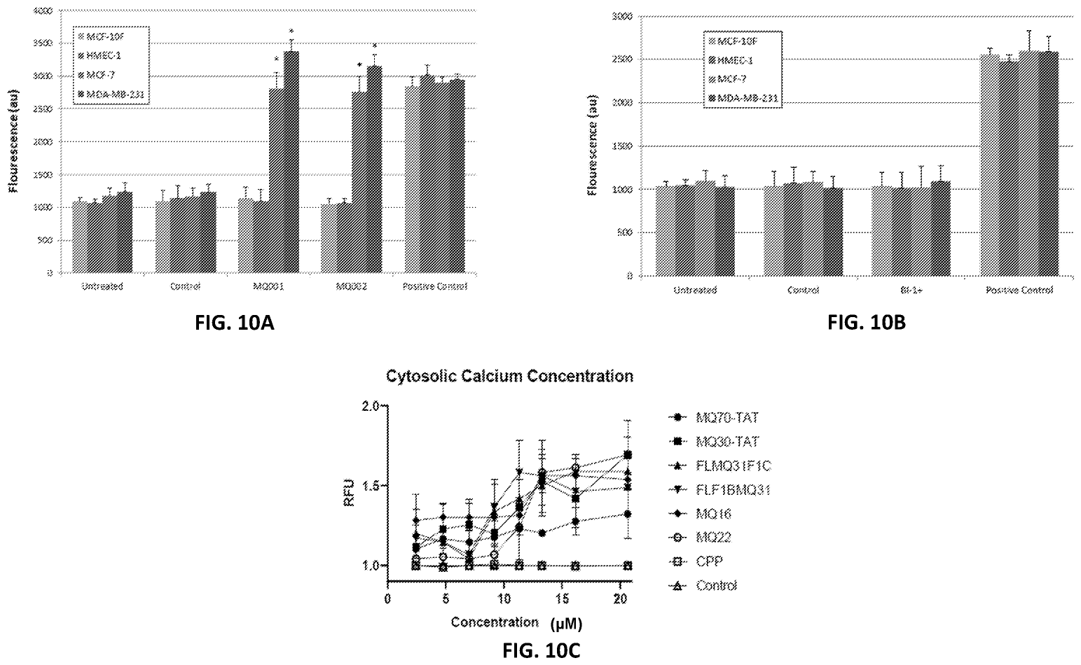

In order to assess the ability of MQ001 and MQ002 to modulate BI-1 regulation of ER calcium concentration and therefore cytosolic calcium concentration, breast cancer cell lines (MCF-7 and MDA-MB-231) as well as non-cancer cell lines (MCF-10F and HMEC-1) were treated for 12 hours with MQ001, MQ002, GFP-CPP (control) or Thapsigargin (positive control) and then incubated with the fluorescent calcium indication Fluo-4 Direct (Invitrogen) for 1 hour. Fluorescent intensity was measured in cells subjected to each treatment condition. Results in FIG. 10A show that treatment with MQ001 and MQ001 significantly increased the cytosolic calcium concentration in breast cancer cells, whereas no increase in cytosolic calcium was observed in non-cancerous cells treated with MQ001 and MQ002. The data thus demonstrate that the interaction of MQ001 or MQ002 with BI-1 does not auto-induce intracellular calcium release as neither non-cancerous cells (FIG. 10A) nor PC3 prostate cancer cells (data not shown) exhibit increase in cytosolic calcium levels following treatment with MQ001 or MQ002.

Breast cancer cells and non-cancerous cells were transfected with BI-1 to induce overexpression of BI-1 and cytosolic calcium levels were subsequently measured in the cells in the absence of treatment with BI-1 modulating peptide. Results in FIG. 10B show that overexpression of BI-1 alone is not sufficient to induce intracellular calcium release in either breast cancer or non-cancerous cells.

To evaluate the relative change in cytosolic calcium concentration induced by treatment with various BI-1 modulating peptides, breast cancer cells were incubated for 19 hours with various BI-1 modulating peptides as well as GFP-CPP (control) and Thapsigargin (positive control) and then assessed for cytosolic calcium levels as described above. Results in FIG. 10C show that increasing concentrations of the BI-1 modulating used to treat cells resulted in increased intracellular calcium release and thus increased cytosolic calcium levels.

### Example 7: Treatment with Exemplary BI-1 Modulating Peptides Results in Production of Reactive Oxygen Species (ROS) in Cancer Cells

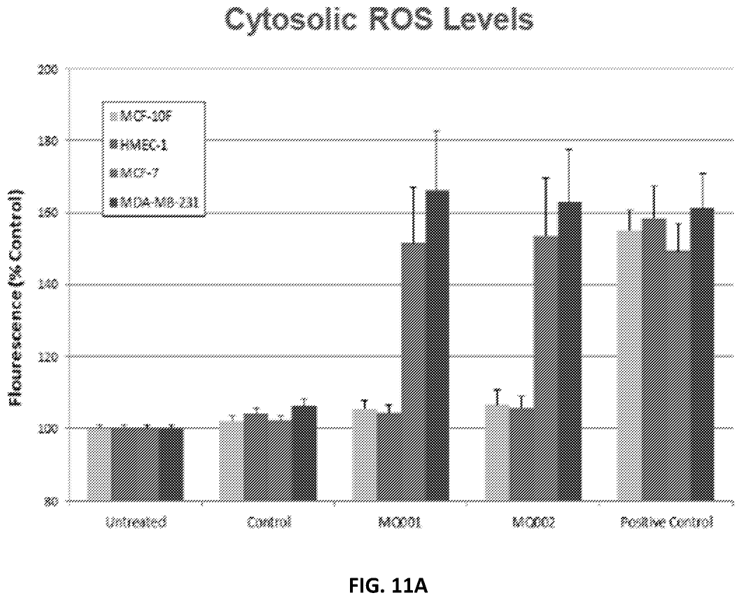

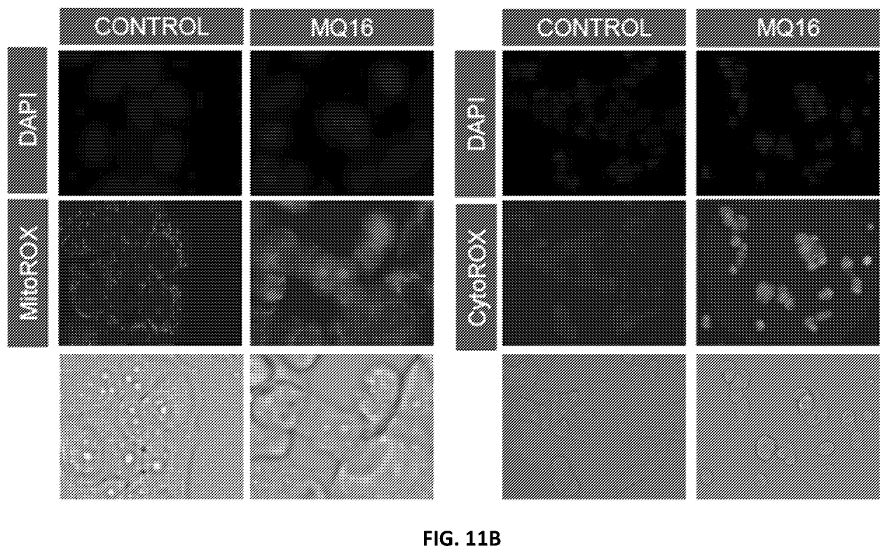

In order to evaluate whether intracellular calcium release in cells treated with BI-1 modulating peptides was accompanied by increased production of ROS, breast cancer cells (MCF-7 and MDA-MB-231) and non-cancerous cells (MCF-10F and HMEC-1) were treated with MQ001, MQ002, or GFP-CPP (control) for 24 hours and then assessed for intracellular levels of ROS using the NBT assay. Results in FIG. 11A show that similar to the increase in cytosolic calcium levels in breast cancer cells following treatment with MQ001 or MQ002, the cytosolic levels of ROS are similarly increased in breast cancer cells following treatment with MQ001 or MQ002. As with cytosolic calcium, the cytosolic ROS levels in non-cancerous cells treated with MQ001 or MQ002 remain unchanged (FIG. 11A)

To further assess production of ROS following treatment with the BI-1 modulating peptide MQ16, MCF-7 cells were treated for 8 hours with or without MQ16 and then stained with CellRox Green Reagent (Thermo Fisher) to probe for induction of oxidative stress. The weakly fluorescent dye in CellRox exhibits bright green photostable fluorescence upon oxidation by ROS. Results in FIG. 11B (right panel) show that oxidative stress was induced and elevated levels of ROS were present in MCF-7 cells following treatment with MQ16.

The presence of superoxides following treatment with MQ16 was assessed using mitochondria-targeting MitoSox Red reagent (Thermo Fisher). The red fluorescent dye in MitoSox is oxidized by superoxides but not by other ROS and reactive nitrogen species (RNS). The oxidized product is highly fluorescent in viable mitochondria. MCF-7 cells were treated with or without MQ16 for 8 hours and then stained with MitoSox Red. Results in FIG. 11B (left panel) show red fluorescent mitochondria in control cells not treated with a BI-1 modulating peptide, indicating the presence of superoxide. Cells treated with MQ16 show diffuse red fluorescence, consistent with permeabilization of mitochondrial membranes and cell lysis. The results demonstrate that treatment of cancer cells with BI-1 modulating peptides induces loss of viable mitochondria.

### Example 8: Treatment with Exemplary BI-1 Modulating Peptides Results in Permeabilization of Mitochondrial Membranes in Cancer Cells

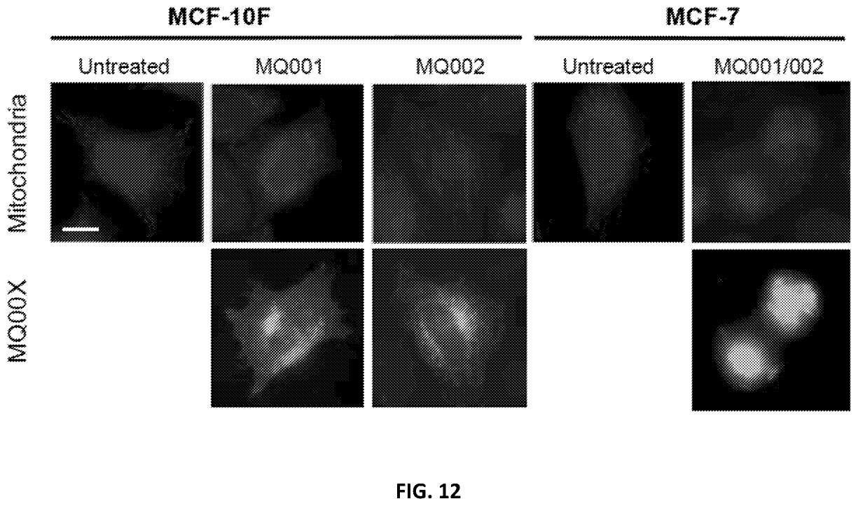

In order to assess the integrity of mitochondrial membranes in cells treated with MQ001 and MQ002, cells from the breast cancer cell line MCF-7 and from the non-cancerous breast cell line MCF-10F were treated and with either HA-tagged MQ001 or HA-tagged MQ002. Cells were incubated with MitoTracker to stain for viable mitochondria (FIG. 12, top) and a fluorescent-tagged anti-HA antibody (FIG. 12, bottom). Results in FIG. 12 show intact mitochondria in MCF-10F cells treated with MQ001 or MQ002 whereas treatment of the MCF-7 breast cancer cells with MQ001 resulted in permeabilization of mitochondrial membranes in the cells.

### Example 9: Treatment with Exemplary BI-1 Modulating Peptides Results in Reorganization of Actin and Distortion of the Endoplasmic Reticulum (ER) in Cancer Cells

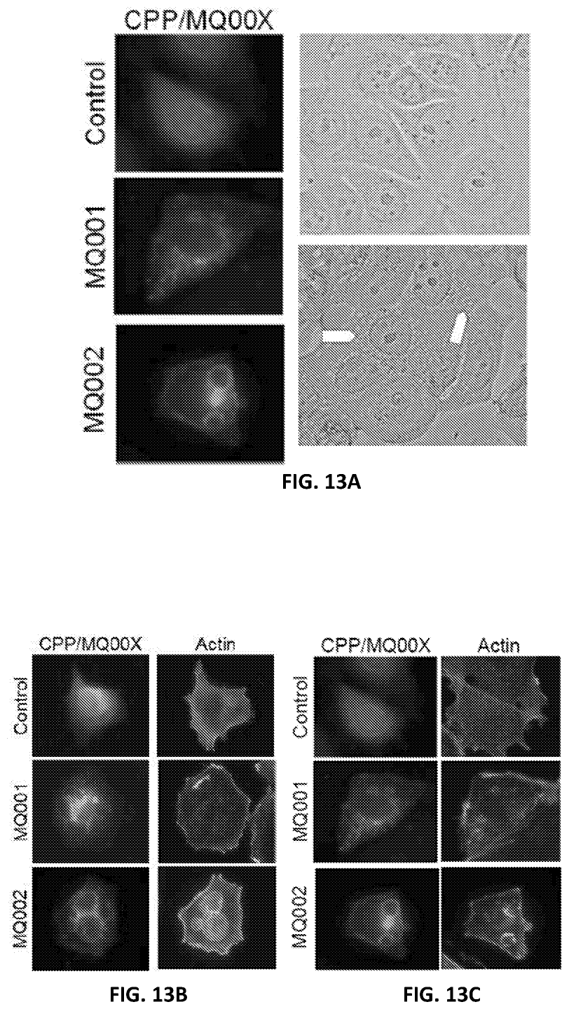

In order to assess whether the BI-1 modulating peptides MQ001 and MQ002 disrupt actin dynamics, MCF-7 cells were treated with Myc-tagged MQ001 or Myc-tagged MQ002 and stained for actin and Myc using antibodies conjugated to fluorophores. Images taken by immunofluorescent microscopy (FIGS. 13C-D) show that actin localization appears to be disrupted in cells treated with MQ001 and MQ002 compared with control. Actin appears to co-localize with MQ001 and MQ002 suggesting that treatment of MCF-7 cells with MQ001 and MQ002 may cause disruption of the cells' actin dynamics, resulting in collapse of the cell structure. Phase-contrast microscopy images (FIG. 13A, right panel) show that the ER appears distorted in MCF-7 cells treated with MQ001 and MQ002 compared to control. Further, immunofluorescent images of the cells show that the MQ001 and MQ002 peptides appear to localize at the ER in treated cells.

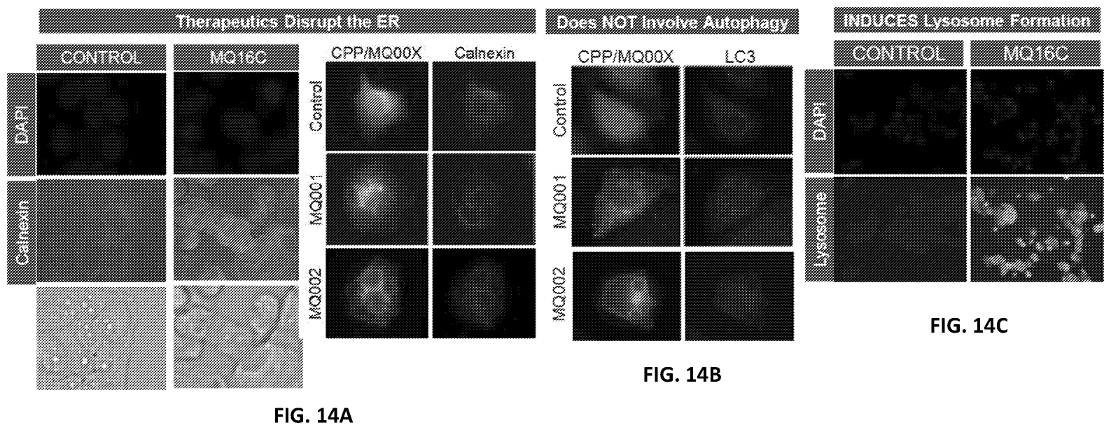

Additional experiments to assess cell morphology and protein localization were performed as above. In some studies cells were also stained with the ER marker calnexin, a lysosome marker, and an antibody to label the autophagy mediating protein LC3. Results show that MQ001 and MQ002 appear to disrupt the structure of the ER compared to control cells, and, further, that both MQ001 and MQ002 appear to localize to the ER in MCF-7 cells (FIG. 14A).

In order to assess whether cell death in cancer cells treated with BI-1 modulating peptides is mediated by autophagy, cells were fluorescently probed using an anti-microtubule-associated protein 1 light chain 3 (LC3) antibody. LC3 is involved in autophagosome formation during autophagy. Results in FIG. 14B show that LC3 is diffuse throughout cells following treatment with a control (CPP) as well following treatment with MQ001 and MQ002. LC3 is not localized to autophagosomes in cancer cells following treatment with the BI-1 modulating peptides, indicating that cell death induced by MQ001 and MQ002 is not mediated by autophagy in these cells. Additional images of stained cells show that treatment with the BI-1 modulating peptide MQ16C induces formation of lysosomes in MCF-7 cells (FIG. 14C).

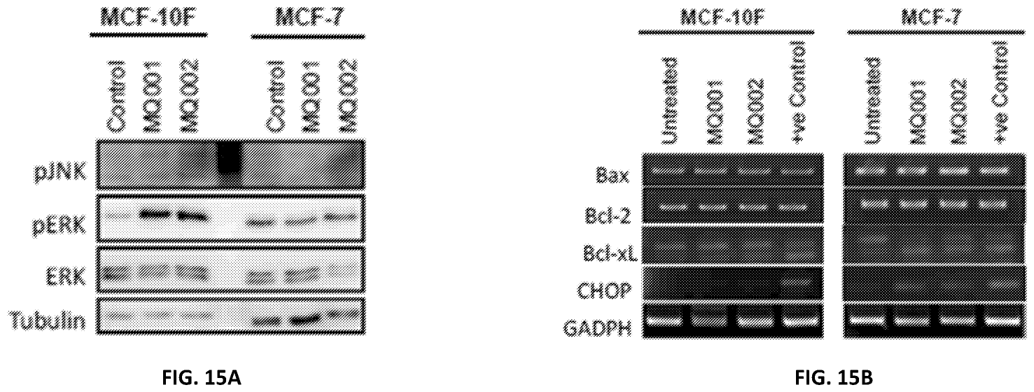

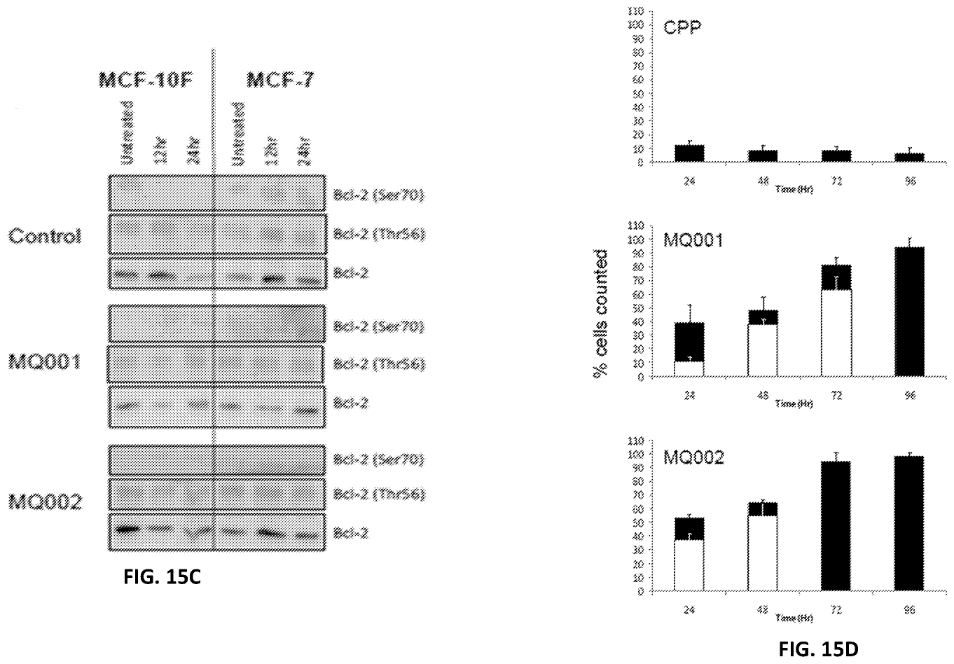

To evaluate whether treatment with the BI-1 modulating peptides MQ001 and MQ002 induce cell death in cancer cells via the ER stress response, known as the unfolded protein response (UPR), lysates from cells treated with MQ001 and MQ002 were assessed by Western blot for increase in phosphorylation of JNK, ERK1/2 and Bcl-2. Activation of the UPR induced transcription factor CHOP was further evaluated by PCR. Results demonstrated that treatment with MQ001 and MQ002 did not result in increased phosphorylation of JNK (FIG. 15A) and only minor activation of CHOP (FIG. 15B). MQ001 and MQ002 induced ERK1/2 phosphorylation in MCF-10F cells (FIG. 15A), which has been shown to be important in promoting cell survival by down-regulating ROS production and inhibiting mitochondrial permeabiliziation (Kim, et al., Biochim Biophys Acta 1823: 876-888, 2012). Elevated ERK phosphorylation was not observed in MCF-7 cells following treatment with MQ001 or MQ002 (FIG. 15A).

In order to confirm that MQ001 and MQ002 prevent activation of the UPR, upregulation of anti-apoptotic genes mediated by IRE1 splicing of XBP-1, a UPR transcription factor that upregulates anti-apoptotic Bcl-2 to inhibit apoptosis, were assessed. MQ001 and MQ002 treatment did not result in any change in Bcl-2 or Bcl-xL expression (FIG. 15B), supporting the idea that MQ001 and MQ002 inhibit IRE1 in cancer cells. Further, no increase or decrease in phosphorylation of Bcl-2 or Bcl-xL was observed following treatment with MQ001 or MQ002 (FIG. 15C).

MCF-7 cells with disrupted ER morphology following treatment with MQ001 or MQ002 as well as MCF-7 cells with condensed nuclei following treatment with MQ001 or MQ002 (both over a 96 hour time course) were counted. FIG. 15D shows quantification of ER disruption (white bars), overlaid onto counts of cells with nuclear condensation (black bars), as counted by immunofluorescence. Cells treated with MQ001 and MQ002 exhibited similar rates of ER degradation and had significantly higher nuclear condensation than the control, both of which increased over time (FIG. 15D).

### Example 10: Treatment with Exemplary BI-1 Modulating Peptides Decreased Tumor Size and Volume by More than 95% in Mouse Models of Human Breast Cancer

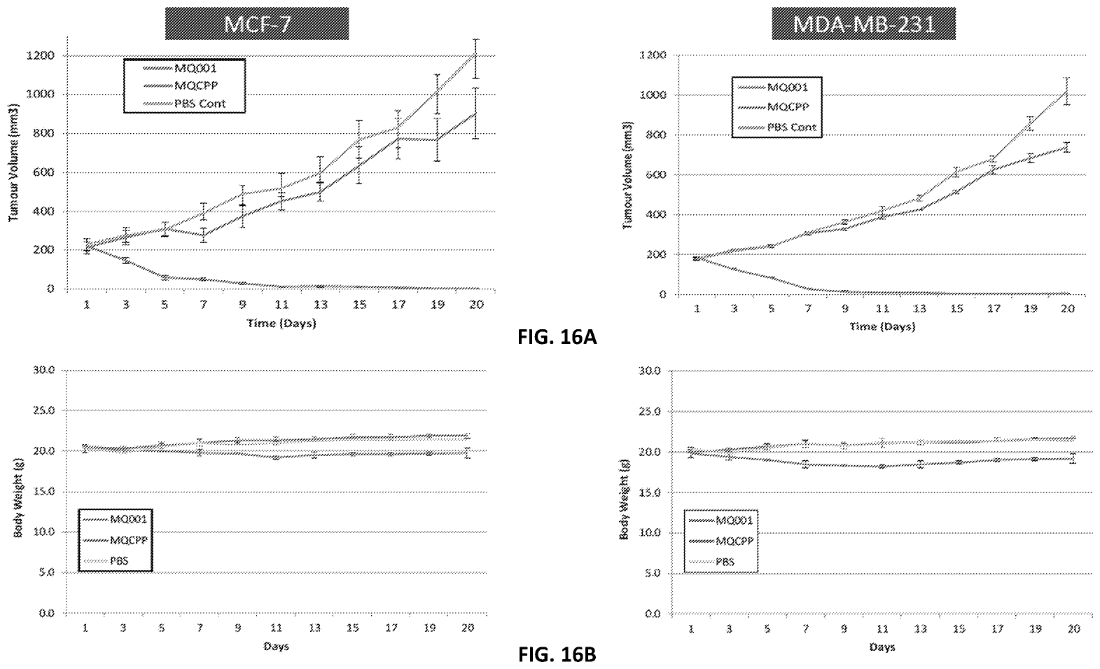

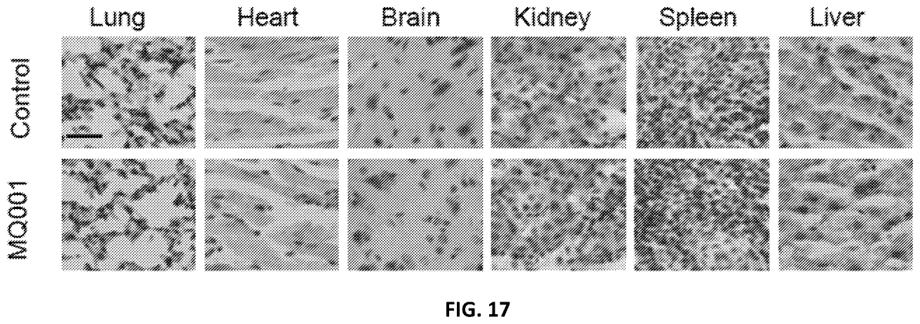

To evaluate the role of BI-1 in breast cancer survival and tumorigenesis, either luminal A MCF-7 or basal MDA-MB-231 human breast carcinoma cells were injected into 8-week old balb/c female mice. Following a growth period to enable the primary tumor to establish in the mice, the tumors were treated with either MQ001, a control, or a placebo for 5 days. Tumors treated with MQ001 were significantly reduced in tumor size and volume within 20 days of treatment (FIG. 16A). Mice treated with MQ001 initially lost weight but largely recovered the weight loss over the period of treatment (FIG. 16B). Toxicity of the major organs were evaluated by H & E staining following MQ001 treatment. No hemorrhage or other indications of toxicity were observed in any of the major organs (FIG. 17).

### Example 11: Stability Assessment of Exemplary BI-1 Modulating Peptides

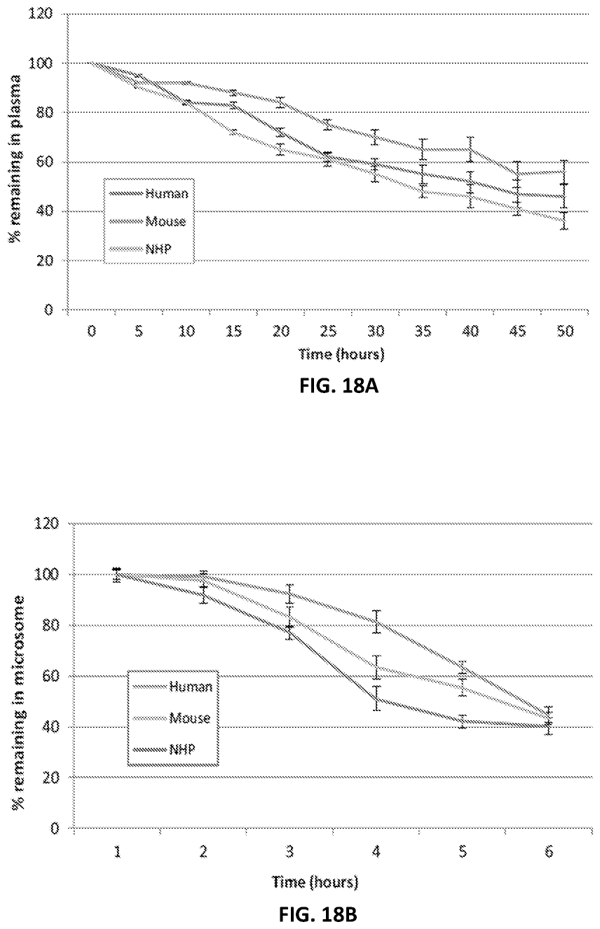

The stability of MQ001 in human, mouse, and non-human primate (NHP) plasma was evaluated in vitro according to the methods described herein. Results demonstrate that approximately 50% of MQ001 remains intact following incubation in human plasma for 50 hours (FIG. 18A).

Stability of MQ001 was also assessed in microsomes according to the methods described herein. Results demonstrate that approximately 40% of MQ001 remains intact in microsomes after 6 hours (FIG. 18B).

### 9. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### 10. INFORMAL SEQUENCE LISTING

| SEQ | | |
|---|---|---|
| ED | | |
| NO | Description | Sequence |
| 1 | NIeH1, full | MLSPSSVNLGCSWNSLTRNLISPDSRILSSVRDAAASSDNGAQVKVGNRT |
| | length | YRVVVTDNKFCVIRESHSGCFTNLLHRLGWPKGEISRKIEVMLNSSPVNR |
| | GenBank: | AMERGAVHSNRPDLPPVDYAPPELPSVDYNSLPVPGNVIGKGGNAVVYED |
| | AIF92440.1 | AEDATKVLKMFTTSQSNEEVINEVRCFNQYYGAGSAEKIYGDNGDIIGIR |
| | | MDKINGESLLNISSLPAQAEHAIYDMFDRLEQKGILFIDTTETNVLYDRT |
| | | RNEFNPIDISSYNISERSWSENQIMQSYHGGKQDLISVVLSKI |
| 2 | NIeH1, C- | NVIGKGGNAVVYEDAEDATKVLKMFTTSQSNEEVTNEVRCFNQYYGAGSA |
| | terminal | EKIYGDNGDIIGIRMDKINGESLLNISSLPAQAEHAIYDMFDRLEQKGIL |
| | | FIDTTEI [symbol] [symbol] |
| | | SVVLSKI |
| 3 | NIeH1, C- | [symbol] [symbol] |
| | terminal | |
| 4 | NIeH2, full | MLSPSSINLGCSWNSLTRNLISPDNRVLSSVRDAAVHSDSGTQVIVGNRT |
| | length | YRVVVTDNKFCVIRESHSGCFTNLLHRLGWPKGEISRKIEAMLNTSPVST |
| | GenBank: | TIERGSVHSNRPDLPPVDYAQPELPPADYTQSELPRVSNNKSPVPGNVIG |
| | AIF93293.1 | KGGNAVVYEDMEDITKVLKMFTISQSHEEVISEVRCFNQYYGSGSAEKIY |
| | | NDNGNVIGIRMNKINGESLLDIPSLPAQAEQAIYDMFDRLEKKGILFVDT |
| | | TETNVLYDRMRNEFNPIDISSYNVSDISWSEHQVMQSYHGGKLDLISVVL |
| | | SKI |
| 5 | NIeH2, C- | NVIGKGGNAVVYEDMEDITKVLKMFTISQSHEEVISEVRCFNQYYGSGSA |
| | terminal | EKIYNDNGNVIGIRMNKINGESLLDIPSLPAQAEQAIYDMFDRLEKKGIL |
| | | FVDTTETNVLYDRMRNEFNPIDISSYNVSDISWSEHQVMQSYHGGKLDLI |
| | | SVVLSKI |
| 6 | NIeH2, C- | NVLYDRMRNEFNPIDISSYNVSDISWSEHQVMQSYHGGKLDLISVVLSKI |
| | terminal | |
| 7 | Ephrin-B2, | MAVRRDSVWKYCWGVLMVLCRTAISKSIVLEPIYWNSSNSKFLPGQGLVL |
| | human | YPQIGDKLDIICPKVDSKTVGQYEYYKVYMVDKDQADRCTIKKENTPLLN |
| | NCBI Ref: | CAKPDQDIKFTIKFQEFSPNLWGLEFQKNKDYYIISTSNGSLEGLDNQEG |
| | NP_004084.1 | GVCQTRAMKILMKVGQDASSAGSTRNKDPIRRPELEAGINGRSSITSPFV |
| | | KPNPGSSIDGNSAGHSGNNILGSEVALFAGIASGCIIFIVIIITLVVLLL |
| | | KYRRRHRKHSPQHTTILSLSTLATPKRSGNNNGSEPSDIIIPLRTADSVF |
| | | CPHYEKVSGDYGHPVYIVQEMPPQSPANIYYKV |
| 8 | Azurin, | MLRKLAAVSLLSLLSAPLLAAECSVDIQGNDQMQFNINAITVDKSCKQFT |
| | Pseudomonas | VNLSHPGNLPKNVMGHNWVLSTAADMQGVVTDGMASGLDKDYLKPDDSRV |
| | aeruginosa | IAHTKLIGSGEKDSVIFDVSKLKEGEQYMFFCTFPGHSALMKGILTLK |
| | NCBI Ref: | |
| | AAA25730.1 | |
| 9 | Azurin p28 | LSTAADMQGVVTDGMASGLDKDYLKPDD |
| 10 | WLSR001 | MLSTAADMQGVVTDGMASGLDKDYLKPDDPRENFGSEFMLSPSSVNLGCS |
| | | WNSLTRNLTSPDSRILSSVRDAAASSDNGAQVKVGNRTYRVVVTDNKFCV |
| | | TRESHSGCFTNLLHRLGWPKGEISRKIEVMLNSSPVNRAMERGAVHSNRP |
| | | DLPPVDYAPPELPSVDYNSLPVPGNVIGKGGNAVVYEDAEDATKVLKMFT |
| | | TSQSNEEVTNEVRCFNQYYGAGSAEKIYGDNGDIIGIRMDKINGESLLNI |
| | | SSLPAQAEHAIYDMFDRLEQKGILFIDTTETNVLYDRTRNEFNPIDISSY |
| | | NISERSWSENQIMQSYHGGKQDLISVVLSKI |
| 11 | BI-1, human | MNIFDRKINFDALLKFSHITPSTQQHLKKVYASFALCMFVAAAGAYVHMV |
| | isoform 1 | THFIQAGLLSALGSLILMIWLMATPHSHETEQKRLGLLAGFAFLTGVGLG |
| | NCBI Ref: | PALEFCIAVNPSILPTAFMGTAMIFTCFTLSALYARRRSYLFLGGILMSA |
| | NP_003208.2 | LSLLLLSSLGNVFFGSIWLFQANLYVGLVVMCGFVLFDTQLIIEKAEHGD |
| | | QDYIWHCIDLFLDFITVFRKLMMILAMNEKDKKKEKK |
| 12 | BI-1, | MNIFDRKINFDALLKFSHITPSTQQHLKKVYASFALCMFV |
| | human, N-terminal | |
| | 40AA | |
| 13 | BI-1, | MNIFDRKINFDALLKFSHITPSTQQHLKK |
| | human, N- | |
| | terminal | |
| | cytoplasmic | |
| | domain | |
| 14 | SboH, | MNISSSGINISTIPTQVKKSVETIRERTKNWFSSEIISVKNTPIFLNEKF |
| | Salmonella | KIGKDSPIEFALPQKIKEFFHPKDKNTLNKTLITVKNITDINNTCKKNIS |
| | bongori | EEVASKMTTAFMRKHIANQSYDYNYRVTSADLLSGGVSISANNRLTVSEG |
| | GenBank | KRDLTSPDANTLSSIQSAVSHSTEGAQVAVGNRTYSVVELNNHFHVSQES |
| | CCC30955.1 | GDNCLMNFLYRPGWPKGEVIRKIELVMNTPRLEINPVKNKTILDKTPGQD |
| | | EMPPIPQVDYNATLHKGEIVGKGGDAIVYADKDDETKVLKMFTIPQLHEE |
| | | VVHEVECFNTYYGKGSAEIIYSNNDISGIKMTRIQGEPVIYAENLPPHAE |
| | | QAIYDMFDRLERNNILFVDTTETNVLYDRDINRFNPIDISSYNLKHTDSK |
| | | DRQDSIIESYICGKSYLINTVLNKIE |
| 15 | OspG, | MKITSTIIQTPFPFENNNSHAGIVTEPILGKLIGQGSTAEIFEDVNDSSA |
| | Shigella | LYKKYDLIGNQYNEILEMAWQESELFNAFYGDEASVVIQYGGDVYLRMLR |
| | flexneri | VPGTPLSDIDTADIPDNIESLYLQLICKLNELSIIHYDLNIGNMLYDKES |
| | GenBank | ESLFPIDFRNIYAEYYAATKKDKEIIDRRLQMRINDFYSLLNRKYL |
| | AAW64846.1 | |
| 16 | MQ001 | MLSTAADMQGVVTDGMASGLDKDYLKPDDSPSSVNLGCSWNSLTRNLISP |
| | | DSRILSSVRDAAASSDNGAQVKVGNRTYRVVVTDNKFCVTRESHSGCFTN |
| | | LLHRLGWPKGEISRKIEVMLNSSPVNRAMERGAVHSNRPDLPPVDYAPPE |
| | | LPSVDYNSLPVPGNVIGKGGNAVVYEDAEDATKVLKMFTTSQSNEEVTNE |
| | | VRCFNQYYGAGSAEKIYGDNGDIIGIRMDKINGESLLNISSLPAQAEHAI |
| | | YDMFDRLEQKGILFIDTTETNVLYDRTRNEFNPIDISSYNISERSWSENQ |
| | | IMQSYHGGKQDLISVVLSKI |
| 17 | MQ157 | NVIGKGGNAVVYEDAEDATKVLKMFTTSQSNEEVTNEVRCFNQYYGAGSA |
| | | EKIYGDNGDIIGIRMDKINGESLLNISSLPAQAEHAIYDMFDRLEQKGIL |
| | | FIDTTETNVLYDRTRNEFNPIDISSYNISERSWSENQIMQSYHGGKQDLI |
| | | SVVLSKI |
| 18 | MQ70 | ASMAQAEHAIYDMFDRLEQKGILFIDTTEINVLYDRTRNEFNPIDISSYN |
| | | ISERSWSENQIMQSYHGGKQDLISVVLSKI |
| 19 | MQ30 | ISERSWSENQIMQSYHGGKQDLISVVLSKI |
| 20 | MQ22 | NQIMQSYHGGKQDLISVVLSKI |
| 21 | MQ16 | NQIMQSYHGGKQDLI |
| 22 | MQ8A | NQIMQSYH |
| 23 | MQ8B | HGGKQDLIS |
| 24 | MQ45 | RTRNEFNPIDISSYNISERSWSENQIMQSYHGGKQDLISVVLSKI |
| 25 | MQ60 | GILFIDTTETNVLYDRTRNEFNPIDISSYNISERSWSENQIMQSYHGGKQ |
| | | DLISVVLSKI |
| 26 | NIeH1(AKA) | MLSPSSVNLGCSWNSLTRNLTSPDSRILSSVRDAAASSDNGAQVKVGNRT |
| | | YRVVVTDNKFCVTRESHSGCFTNLLHRLGWPKGEISRKIEVMLNSSPVNR |
| | | AMERGAVHSNRPDLPPVDYAPPELPSVDYNSLPVPGNVIGKGGNAVVYED |
| | | AEDATKVLKMFTTSQSNEEVTNEVRCFNQYYGAGSAEKIYGDNGDIIGIR |
| | | MDKINGESLLNISSLPAQAEHAIYDMFDRLEQKGILFIDTTETNVLYDRT |
| | | RNEFNPIDISSYNISERSWSENQIMQSYHGGKQDLISVVLAKA |
| 27 | MQ002 | MLSPSSINLGCSWNSLTRNLTSPDNRVLSSVRDAAVHSDSGTQVTVGNRT |
| | & | YRVVVTDNKFCVTRESHSGCFTNLLHRLGWPKGEISRKIEAMLNTSPVST |
| | NIeH2(AKA) | TIERGSVHSNRPDLPPVDYAQPELPPADYTQSELPRVSNNKSPVPGNVIG |
| | | KGGNAVVYEDMEDITKVLKMFTISQSHEEVTSEVRCFNQYYGSGSAEKIY |
| | | NDNGNVIGIRMNKINGESLLDIPSLPAQAEQAIYDMFDRLEKKGILFVDT |
| | | TETNVLYDRMRNEFNPIDISSYNVSDISWSEHQVMQSYHGGKLDLISVVL |
| | | AKA |
| 28 | 8 AA CPP | VATDGMPAG |
| | (9 aa) | |
| 29 | 12 AA CPP | AGADNAFLKAGD |
| | (12 aa) | |
| 30 | 18 AA CPP | LTKAADMAGVATDGMPAG |
| | (18 aa) | |
| 31 | 28 AA CPP | LTKAADMAGVATDGMPAGADNAFLKAGD |
| | (28 aa) | |
| 32 | MQ157 + N- | VATDGMPAGNVIGKGGNAVVYEDAEDATKVLKMFTTSQSNEEVTNEVRCF |
| | term. 8AA | NQYYGAGSAEKIYGDNGDIIGIRMDKINGESLLNISSLPAQAEHAIYDMF |
| | CPP | DRLEQKGILFIDTTETNVLYDRTRNEFNPIDISSYNISERSWSENQIMQS |
| | | YHGGKQDLISVVLSKI |
| 33 | MQ157 +C- | NVIGKGGNAVVYEDAEDATKVLKMFTTSQSNEEVTNEVRCFNQYYGAGSA |
| | term. 8AA | EKIYGDNGDIIGIRMDKINGESLLNISSLPAQAEHAIYDMFDRLEQKGIL |
| | CPP | FIDTTETNVLYDRTRNEFNPIDISSYNISERSWSENQIMQSYHGGKQDLI |
| | | SVVLSKIVATDGMPAG |
| 34 | MQ157 + N- | AGADNAFLKAGDNVIGKGGNAVVYEDAEDATKVLKMFTTSQSNEEVTNEV |
| | term. 12AA | RCFNQYYGAGSAEKIYGDNGDIIGIRMDKINGESLLNISSLPAQAEHAIY |
| | CPP | DMFDRLEQKGILFIDTTETNVLYDRTRNEFNPIDISSYNISERSWSENQI |
| | | MQSYHGGKQDLISVVLSKI |
| 35 | MQ157 +C- | NVIGKGGNAVVYEDAEDATKVLKMFTTSQSNEEVTNEVRCFNQYYGAGSA |
| | term. 12AA | EKIYGDNGDIIGIRMDKINGESLLNISSLPAQAEHAIYDMFDRLEQKGIL |
| | CPP | FIDTTETNVLYDRTRNEFNPIDISSYNISERSWSENQIMQSYHGGKQDLI |
| | | SVVLSKIAGADNAFLKAGD |
| 36 | MQ157 + N- | LTKAADMAGVATDGMPAGNVIGKGGNAVVYEDAEDATKVLKMFTTSQSNE |
| | term. 18AA | EVTNEVRCFNQYYGAGSAEKIYGDNGDIIGIRMDKINGESLLNISSLPAQ |
| | CPP | AEHAIYDMFDRLEQKGILFIDTTETNVLYDRTRNEFNPIDISSYNISERS |
| | | WSENQIMQSYHGGKQDLISVVLSKI |
| 37 | MQ157 +C- | NVIGKGGNAVVYEDAEDATKVLKMFTTSQSNEEVTNEVRCFNQYYGAGSA |
| | term. 18AA | EKIYGDNGDIIGIRMDKINGESLLNISSLPAQAEHAIYDMFDRLEQKGIL |
| | CPP | FIDTTETNVLYDRTRNEFNPIDISSYNISERSWSENQIMQSYHGGKQDLI |
| | | SVVLSKILTKAADMAGVATDGMPAG |
| 38 | MQ157 + N- | LTKAADMAGVATDGMPAGADNAFLKAGDNVIGKGGNAVVYEDAEDATKVL |
| | term. 28AA | KMFTTSQSNEEVTNEVRCFNQYYGAGSAEKIYGDNGDIIGIRMDKINGES |
| | CPP | LLNISSLPAQAEHAIYDMFDRLEQKGILFIDTTETNVLYDRTRNEFNPID |
| | | ISSYNISERSWSENQIMQSYHGGKQDLISVVLSKI |
| 39 | MQ157 + C- | NVIGKGGNAVVYEDAEDATKVLKMFTTSQSNEEVTNEVRCFNQYYGAGSA |
| | term. 28AA | EKIYGDNGDIIGIRMDKINGESLLNISSLPAQAEHAIYDMFDRLEQKGIL |
| | CPP | FIDTTETNVLYDRTRNEFNPIDISSYNISERSWSENQIMQSYHGGKQDL |
| | | ISVVLSKILTKAADMAGVATDGMPAGADNAFLKAGD |
| 40 | MQ70 + N- | VATDGMPAGASMAQAEHAIYDMFDRLEQKGILFIDTTETNVLYDRTRNEF |
| | term. 8AA | NPIDISSYNISERSWSENQIMQSYHGGKQDLISVVLSKI |
| | CPP | |
| 41 | MQ70 + C- | ASMAQAEHAIYDMFDRLEQKGILFIDTTETNVLYDRTRNEFNPIDISSYN |
| | term. 8AA | ISERSWSENQIMQSYHGGKQDLISVVLSKIVATDGMPAG |
| | CPP | |
| 42 | MQ70 + N- | AGADNAFLKAGDASMAQAEHAIYDMFDRLEQKGILFIDTTETNVLYDRTR |
| | term. 12AA | NEFNPIDISSYNISERSWSENQIMQSYHGGKQDLISVVLSKI |
| | CPP | |
| 43 | MQ70 + C- | ASMAQAEHAIYDMFDRLEQKGILFIDTTETNVLYDRTRNEFNPIDISSYN |
| | term. 12AA | ISERSWSENQIMQSYHGGKQDLISVVLSKIAGADNAFLKAGD |
| | CPP | |
| 44 | MQ70 + N- | LTKAADMAGVATDGMPAGASMAQAEHAIYDMFDRLEQKGILFIDTTETNV |
| | term. 18AA | LYDRTRNEFNPIDISSYNISERSWSENQIMQSYHGGKQDLISVVLSKI |
| | CPP | |
| 45 | MQ70 + C- | ASMAQAEHAIYDMFDRLEQKGILFIDTTETNVLYDRTRNEFNPIDISSYN |
| | term. 18AA | ISERSWSENQIMQSYHGGKQDLISVVLSKILTKAADMAGVATDGMPAG |
| | CPP | |
| 46 | MQ70 + N- | LTKAADMAGVATDGMPAGADNAFLKAGDASMAQAEHAIYDMFDRLEQKGI |
| | term. 28AA | LFIDTTETNVLYDRTRNEFNPIDISSYNISERSWSENQIMQSYHGGKQDL |
| | CPP | ISVVLSKI |
| 47 | MQ70 + C- | ASMAQAEHAIYDMFDRLEQKGILFIDTTETNVLYDRTRNEFNPIDISSYN |
| | term. 28AA | ISERSWSENQIMQSYHGGKQDLISVVLSKILTKAADMAGVATDGMPAGAD |
| | CPP | NAFLKAGD |
| 48 | MQ30 + N- | VATDGMPAGISERSWSENQIMQSYHGGKQDLISVVLSKI |
| | term. 8AA | |
| | CPP & | |
| | F1NMQ30 | |
| 49 | MQ30 + C- | ISERSWSENQIMQSYHGGKQDLISVVLSKIVATDGMPAG |
| | term. 8AA | |
| | CPP & | |
| | MQ30F1C | |
| 50 | MQ30 + N- | AGADNAFLKAGDISERSWSENQIMQSYHGGKQDLISVVLSKI |
| | term. 12AA | |
| | CPP | |
| 51 | MQ30 + C- | ISERSWSENQIMQSYHGGKQDLISVVLSKIAGADNAFLKAGD |
| | term. 12AA | |
| | CPP | |
| 52 | MQ30 + N- | LTKAADMAGVATDGMPAGISERSWSENQIMQSYHGGKQDLISVVLSKI |
| | term. 18AA | |
| | CPP | |
| 53 | MQ30 + C- | ISERSWSENQIMQSYHGGKQDLISVVLSKILTKAADMAGVATDGMPAG |
| | term. 18AA | |
| | CPP | |
| 54 | MQ30 + N- | LTKAADMAGVATDGMPAGADNAFLKAGDISERSWSENQIMQSYHGGKQDL |
| | term. 28AA | ISVVLSKI |
| | CPP | |
| 55 | MQ30 + C- | ISERSWSENQIMQSYHGGKQDLISVVLSKILTKAADMAGVATDGMPAGAD |
| | term. 28AA | NAFLKAGD |
| | CPP | |
| 56 | MQ22 + N- | VATDGMPAGNQIMQSYHGGKQDLISVVLSKI |
| | term. 8AA | |
| | CPP & | |
| | FLF1BMQ31 | |
| 57 | MQ22 + C- | NQIMQSYHGGKQDLISVVLSKIVATDGMPAG |
| | term. 8AA | |
| | CPP & | |
| | FLMQ31F1C | |
| 58 | MQ22 + N- | AGADNAFLKAGDNQIMQSYHGGKQDLISVVLSKI |
| | term. 12AA | |
| | CPP | |
| 59 | MQ22 + C- | NQIMQSYHGGKQDLISVVLSKIAGADNAFLKAGD |
| | term. 12AA | |
| | CPP | |
| 60 | MQ22 + N- | LTKAADMAGVATDGMPAGNQIMQSYHGGKQDLISVVLSKI |
| | term. 18AA | |
| | CPP | |
| 61 | MQ22 + C- | NQIMQSYHGGKQDLISVVLSKILTKAADMAGVATDGMPAG |
| | term. 18AA | |
| | CPP | |
| 62 | MQ22 + N- | LTKAADMAGVATDGMPAGADNAFLKAGDNQIMQSYHGGKQDLISVVLSKI |
| | term. 28AA | |
| | CPP | |
| 63 | MQ22 + C- | NQIMQSYHGGKQDLISVVLSKILTKAADMAGVATDGMPAGADNAFLKAGD |
| | term. 28AA | |
| | CPP & | |
| | MQ22C | |
| 64 | MQ16 + N- | VATDGMPAGNQIMQSYHGGKQDLI |
| | term. 8AA | |
| | CPP | |
| 65 | MQ16 + C- | NQIMQSYHGGKQDLIVATDGMPAG |
| | term. 8AA | |
| | CPP | |
| 66 | MQ16 + N- | AGADNAFLKAGDNQIMQSYHGGKQDLI |
| | term. 12AA | |
| | CPP | |
| 67 | MQ16 + C- | NQIMQSYHGGKQDLIAGADNAFLKAGD |
| | term. 12AA | |
| | CPP | |
| 68 | MQ16 + N- | LTKAADMAGVATDGMPAGNQIMQSYHGGKQDLI |
| | term. 18AA | |
| | CPP | |
| 69 | MQ16 + C- | NQIMQSYHGGKQDLILTKAADMAGVATDGMPAG |
| | term. 18AA | |
| | CPP | |
| 70 | MQ16 + N- | LTKAADMAGVATDGMPAGADNAFLKAGDNQIMQSYHGGKQDLI |
| | term. 28AA | |
| | CPP | |
| 71 | MQ16 + C- | NQIMQSYHGGKQDLILTKAADMAGVATDGMPAGADNAFLKAGD |
| | term. 28AA | |
| | CPP & | |
| | MQ16C | |
| 72 | MQ8A + N- | VATDGMPAGNQIMQSYH |
| | term. 8AA | |
| | CPP | |
| 73 | MQ8A + C- | NQIMQSYHVATDGMPAG |
| | term. 8AA | |
| | CPP | |
| 74 | MQ8A + N- | AGADNAFLKAGDNQIMQSYH |
| | term. 12AA | |
| | CPP | |
| 75 | MQ8A + C- | NQIMQSYHAGADNAFLKAGD |
| | term. 12AA | |
| | CPP | |
| 76 | MQ8A + N- | LTKAADMAGVATDGMPAGNQIMQSYH |
| | term. 18AA | |
| | CPP | |
| 77 | MQ8A + C- | NQIMQSYHLTKAADMAGVATDGMPAG |
| | term. 18AA | |
| | CPP | |
| 78 | MQ8A + N- | LTKAADMAGVATDGMPAGADNAFLKAGDNQIMQSYH |
| | term. 28AA | |
| | CPP | |
| 79 | MQ8A + C- | NQIMQSYHLTKAADMAGVATDGMPAGADNAFLKAGD |
| | term. 28AA | |
| | CPP | |
| 80 | MQ8B + N- | VATDGMPAGHGGKQDLIS |
| | term. 8AA | |
| | CPP | |
| 81 | MQ8B + C- | HGGKQDLISVATDGMPAG |
| | term. 8AA | |
| | CPP | |
| 82 | MQ8B + N- | AGADNAFLKAGDHGGKQDLIS |
| | term. 12AA | |
| | CPP | |
| 83 | MQ8B + C- | HGGKQDLISAGADNAFLKAGD |
| | term. 12AA | |
| | CPP | |
| 84 | MQ8B + N- | LTKAADMAGVATDGMPAGHGGKQDLIS |
| | term. 18AA | |
| | CPP | |
| 85 | MQ8B + C- | HGGKQDLISLTKAADMAGVATDGMPAG |
| | term. 18AA | |
| | CPP | |
| 86 | MQ8B + N- | LTKAADMAGVATDGMPAGADNAFLKAGDHGGKQDLIS |
| | term. 28AA | |
| | CPP | |
| 87 | MQ8B + C- | HGGKQDLISLTKAADMAGVATDGMPAGADNAFLKAGD |
| | term. 28AA | |
| | CPP | |
| 88 | MQ45 + N- | VATDGMPAGRTRNEFNPIDISSYNISERSWSENQIMQSYHGGKQDLISVV |
| | term. 8AA | LSKI |
| | CPP | |
| 89 | MQ45 + C- | RTRNEFNPIDISSYNISERSWSENQIMQSYHGGKQDLISVVLSKIVATDG |
| | term. 8AA | MPAG |
| | CPP | |
| 90 | MQ45 + N- | AGADNAFLKAGDRTRNEFNPIDISSYNISERSWSENQIMQSYHGGKQDLI |
| | term. 12AA | SVVLSKI |
| | CPP | |
| 91 | MQ45 + C- | RTRNEFNPIDISSYNISERSWSENQIMQSYHGGKQDLISVVLSKIAGADN |
| | term. 12AA | AFLKAGD |
| | CPP | |
| 92 | MQ45 + N- | LTKAADMAGVATDGMPAGRTRNEFNPIDISSYNISERSWSENQIMQSYHG |
| | term. 18AA | GKQDLISVVLSKI |
| | CPP | |
| 93 | MQ45 + C- | RTRNEFNPIDISSYNISERSWSENQIMQSYHGGKQDLISVVLSKILTKAA |
| | term. 18AA | DMAGVATDGMPAG |
| | CPP | |
| 94 | MQ45 + N- | LTKAADMAGVATDGMPAGADNAFLKAGDRTRNEFNPIDISSYNISERSWS |
| | term. 28AA | ENQIMQSYHGGKQDLISVVLSKI |
| | CPP | |
| 95 | MQ45 + C- | RTRNEFNPIDISSYNISERSWSENQIMQSYHGGKQDLISVVLSKILTKAA |
| | term. 28AA | DMAGVATDGMPAGADNAFLKAGD |
| | CPP | |
| 96 | MQ60 + N- | VATDGMPAGGILFIDTTETNVLYDRTRNEFNPIDISSYNISERSWSENQI |
| | term. 8AA | MQSYHGGKQDLISVVLSKI |
| | CPP | |
| 97 | MQ60 + C- | GILFIDTTETNVLYDRTRNEFNPIDISSYNISERSWSENQIMQSYHGGKQ |
| | term. 8AA | DLISVVLSKIVATDGMPAG |
| | CPP | |
| 98 | MQ60 + N- | AGADNAFLKAGDGILFIDTTETNVLYDRTRNEFNPIDISSYNISERSWSE |
| | term. 12AA | NQIMQSYHGGKQDLISVVLSKI |
| | CPP | |
| 99 | MQ60 + C- | GILFIDTTETNVLYDRTRNEFNPIDISSYNISERSWSENQIMQSYHGGKQ |
| | term. 12AA | DLISVVLSKIAGADNAFLKAGD |
| | CPP | |
| 100 | MQ60 + N- | LTKAADMAGVATDGMPAGGILFIDTTETNVLYDRTRNEFNPIDISSYNIS |
| | term. 18AA | ERSWSENQIMQSYHGGKQDLISVVLSKI |
| | CPP | |
| 101 | MQ60 + C- | GILFIDTTETNVLYDRTRNEFNPIDISSYNISERSWSENQIMQSYHGGKQ |
| | term. 18AA | DLISVVLSKILTKAADMAGVATDGMPAG |
| | CPP | |
| 102 | MQ60 + N- | LTKAADMAGVATDGMPAGADNAFLKAGDGILFIDTTETNVLYDRTRNEFN |
| | term. 28AA | PIDISSYNISERSWSENQIMQSYHGGKQDLISVVLSKI |
| | CPP | |
| 103 | MQ60 + C- | GILFIDTTETNVLYDRTRNEFNPIDISSYNISERSWSENQIMQSYHGGKQ |
| | term. 28AA | DLISVVLSKILTKAADMAGVATDGMPAGADNAFLKAGD |
| | CPP | |
| 104 | TAT | GRKKRRQRRRPPQ |
| | sequence | |
| 105 | MQ30-TAT | ISERSWSENQIMQSYHGGKQDLISVVLSKIGRKKRRQRRPPQ |
| 106 | MQ70-TAT | ASMAQAEHAIYDMFDRLEQKGILFIDTTETNVLYDRTRNEFNPIDISSYN |
| | | ISERSWSENQIMQSYHGGKQDLISVVLSKIGRKKRRQRRRPPQ |

What is claimed is:

1. An isolated peptide, comprising:
(a) a Bax Inhibitor-1 (BI-1) modulating domain; and, optionally,
(b) a targeting domain.

2. The isolated peptide of claim 1, wherein the BI-1 modulating domain comprises:
a peptide segment having the sequence of SEQ ID NO: 22 or a sequence that differs by no more than one amino acid residue from the sequence of SEQ ID NO: 22; and/or
a peptide segment having the sequence of SEQ ID NO: 23 or a sequence that differs by no more than one amino acid residue from the sequence of SEQ ID NO:23.

3. The isolated peptide of claim 2, wherein the BI-1 modulating domain comprises a peptide segment having the sequence of SEQ ID NO: 22 and/or SEQ ID NO: 23.

4. The isolated peptide of claim 3, wherein the BI-1 modulating domain comprises a peptide segment having the sequence of SEQ ID NO: 22 and a peptide segment having the sequence of SEQ ID NO: 23.

5. The isolated peptide of claim 4, wherein the segment having the sequence of SEQ ID NO: 22 is amino terminal to the segment having the sequence of SEQ ID NO: 23.

6. The isolated peptide of claim 4, wherein the BI-1 modulating domain comprises a segment having the sequence of SEQ ID NO: 22 and SEQ ID NO: 23, wherein the sequence of SEQ ID NO:22 and SEQ ID NO:23 overlap within the segment.

7. The isolated peptide of claim 3, wherein the BI-1 modulating domain has the sequence of SEQ ID NO: 16.

8. The isolated peptide of claim 3, wherein the BI-1 modulating domain has the sequence of SEQ ID NO: 17.

9. The isolated peptide of claim 3, wherein the BI-1 modulating domain has the sequence set of SEQ ID NO: 18.

10. The isolated peptide of claim 3, wherein the BI-1 modulating domain has the sequence of SEQ ID NO: 19.

11. The isolated peptide of claim 3, wherein the BI-1 modulating domain has the sequence of SEQ ID NO: 20.

12. The isolated peptide of claim 3, wherein the BI-1 modulating domain has the sequence of SEQ ID NO: 21.

13. The isolated peptide of claim 3, wherein the BI-1 modulating domain has the sequence of SEQ ID NO: 24.

14. The isolated peptide of claim 3, wherein the BI-1 modulating domain has the sequence of SEQ ID NO: 25.

15. The isolated peptide of claim 3, wherein the BI-1 modulating domain has the sequence of SEQ ID NO: 26.

16. The isolated peptide of claim 3, wherein the BI-1 modulating domain has the sequence of SEQ ID NO: 27.

17. The isolated peptide of any of the preceding claims, wherein the BI-1 modulating domain is capable of binding to a BI-1 protein.

18. The isolated peptide of any of the preceding claims, wherein the BI-1 modulating domain is capable of binding to a site within a BI-1 protein within the amino acid sequence of SEQ ID NO: 13.

19. The isolated peptide of any of the preceding claims, wherein the peptide is capable of being coupled to a liposome.

20. The isolated peptide of any of the preceding claims, wherein the peptide is capable of being conjugated to a nanoparticle

21. The isolated peptide of any of the preceding claims, wherein the targeting domain is a cell penetrating peptide (CPP), an antibody, or a fragment of an antibody.

22. The isolated peptide of any of the preceding claims, wherein the targeting domain is capable of binding a tumor-associated antigen.

23. The isolated peptide of any of the preceding claims, wherein the targeting domain is at the amino terminus of the peptide.

24. The isolated peptide of any of the preceding claims, wherein the targeting domain is at the carboxy terminus of the peptide.

25. The isolated peptide of any of the preceding claims, wherein the peptide is between 5 and 400 amino acids in length.

26. The isolated peptide of any of the preceding claims, wherein the peptide is between 8 and 40 amino acids in length.

27. The isolated peptide of any of the preceding claims, wherein the peptide is between 15 and 45 amino acids in length.

28. The isolated peptide of any of the preceding claims, wherein the peptide is between 22 and 50 amino acids in length.

29. The isolated peptide of any of the preceding claims, wherein the peptide is between 30 and 60 amino acids in length.

30. The isolated peptide of any of the preceding claims, wherein the peptide is between 45 and 75 amino acids in length.

31. The isolated peptide of any of the preceding claims, wherein the peptide is between 60 and 100 amino acids in length.

32. The isolated peptide of any of the preceding claims, wherein the peptide is between 80 and 110 amino acids in length.

33. The isolated peptide of any of the preceding claims, wherein the peptide is between 280 and 320 amino acids in length.

34. The isolated peptide of claim 1, having an amino acid sequence with at least 85% sequence identity to the sequence of any one of SEQ ID NOs: 19-23 and 48-87.

35. The isolated peptide of claim 34, comprising an amino acid sequence with at least 85% sequence identity to the sequence of SEQ ID NO: 19.

36. The isolated peptide of claim 34, comprising an amino acid sequence with at least 85% sequence identity to the sequence of SEQ ID NO: 20.

37. The isolated peptide of claim 34, comprising an amino acid sequence with at least 85% sequence identity to the sequence of SEQ ID NO: 21.

38. The isolated peptide of claim 34, comprising an amino acid sequence with at least 85% sequence identity to the sequence of SEQ ID NO: 22.

39. The isolated peptide of claim 34, comprising an amino acid sequence with at least 85% sequence identity to the sequence of SEQ ID NO: 23.

40. The isolated peptide of any of the preceding claims, wherein the peptide further comprises a chemical modification.

41. The isolated peptide of claim 40, wherein the chemical modification is phosphorylation, glycosylation, and/or lipidation.

42. The isolated peptide of claim 41, wherein the chemical modification is a covalent linkage of a fatty acid.

43. The isolated peptide of claim 40, wherein the chemical modification is a chemical blocking of the terminal amine group.

44. The isolated peptide of claim 40, wherein the chemical modification is a chemical blocking of the terminal carboxy group.

45. The isolated peptide of any of the preceding claims, wherein the peptide further comprises an Fc polypeptide or domain.

46. The isolated peptide of any of the preceding claims, wherein the peptide further comprises a non-peptide linker.

47. The isolated peptide of any of the preceding claims, wherein the peptide is conjugated to one or more PEG molecules.

48. The isolated peptide of any of the preceding claims, wherein the isolated peptide is capable of passing through a plasma membrane of a mammalian cell.

49. The isolated peptide of claim 48, wherein the cell is a human cell.

50. A pharmaceutical composition comprising the peptide of any of the preceding claims and a pharmaceutically acceptable carrier.

51. The pharmaceutical composition of claim 50, wherein the pharmaceutical composition is suitable for parenteral administration.

52. The pharmaceutical composition of claim 51, wherein the pharmaceutical composition is suitable for intravenous administration.

53. The pharmaceutical composition of claim 51, wherein the pharmaceutical composition is suitable for subcutaneous administration.

54. The pharmaceutical composition of any one of claims 47-50, wherein the concentration of active ingredient is 100 nM or greater.

55. The pharmaceutical composition of any one of claims 47-51, wherein the pharmaceutically acceptable carrier is suitable for enhancing solubility of the peptide.

56. The pharmaceutical composition of any one of claims 47-52, wherein the pharmaceutical composition is in a single-dose prefilled syringe.

57. A method of treating a subject having a proliferative disease, the method comprising: administering to the subject an effective amount of the peptide or the pharmaceutical composition of any of the preceding claims.

58. The method of claim 57, wherein the proliferative disease is cancer.

59. The method of claim 58, wherein the cancer is at least one of the group consisting of: breast, ovarian, lung, uterine, and colon cancer.

60. The method of claim 58 or 59, wherein the cancer is breast cancer.

61. The method of any one of claims 57-60, wherein the administering results in an increase in cytosolic calcium levels in cells of the subject.

62. The method of any one of claims 57-61, wherein the administering results in an increase in cytosolic concentration of W ions in cells of the subject.

63. The method of any one of claims 57-62, wherein the administering results in an increase in permeabilization of mitochondrial membranes in neoplastic cells in the subject.

64. The method of any one of claims 57-63, wherein the administering induces death of neoplastic cells in the subject.

65. The method of any one of claims 57-64, wherein the administering induces apoptosis and/or paraptosis of neoplastic cells in the subject.

66. The method of any one of claims 54-65, wherein the peptide or the pharmaceutical composition is administered by intravenous administration.

67. The method of any one of claims 54-65, wherein the peptide or the pharmaceutical composition is administered by subcutaneous administration.

68. The method of any one of claims 57-58, wherein the peptide or the pharmaceutical composition is administered by intrathecal or intra-cisterna magna administration for treatment of brain cancer.

69. The method of any one of claims 57-68, further comprising administering a second effective amount of a further treatment.

70. The method of claim 69, wherein the further treatment is selected from the group consisting of: a chemotherapeutic agent, a radiation treatment, and an antibody or antibody fragment.

71. The method of any one of claims 57-70, wherein the subject is a mammal.

72. The method of claim 71, wherein the subject is a human.

73. An isolated nucleic acid molecule comprising a polynucleotide encoding the peptide of any one of claims 1-49.
